SEQUENCE LISTING (14 sequences)

SEQ ID NO: 1 - protein, 7 aa, synthetic construct
  REGION 1..7: note = Insertion peptide
ISDQTKH

SEQ ID NO: 2 - protein, 10 aa, synthetic construct
  REGION 1..10: note = Insertion peptide
LAISDQTKHA

SEQ ID NO: 3 - protein, 745 aa, synthetic construct
  REGION 1..745: note = Variant AAV capsid
MAADGYLPDWLEDTLSEGIRQWWKLKPGPPPPKAAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNLAISDQTKHARQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - DNA, 1374 bp, synthetic construct
  misc_feature 1..1374: note = codon optimized aflibercept sequence
  source 1..1374: mol_type = other DNA
atggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaa

SEQ ID NO: 5 - protein, 458 aa, synthetic construct
  REGION 1..458: note = aflibercept sequence
MVSYWDTGVLLCALLSCLLLTGSSSGSDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 3834 bp, synthetic construct
  misc_feature 1..3834: note = synthetic polynucleotide
  source 1..3834: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcgcgacttcttaacccaacagaaggctcgagaaggtatattgctgttgacagtgagcggctacctcagcaagacgttatttagtgaagccacagatgtaaataacgtcttgctgaggtagctgcctactgcctcggacttcaaggggctagaattcgcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttggctagcacgccaccatggtttcttactgggacaccggcgtgctgctgtgtgccctgctttcttgtctgctgctgaccggctctagcagcggctctgataccggcagacccttcgtggaaatgtacagcgagatccccgagatcatccacatgaccgagggcagagagctggtcatcccttgcagagtgacaagccccaacatcaccgtgactctgaagaagttccctctggacacactgatccccgacggcaagagaatcatctgggacagccggaagggcttcatcatcagcaacgccacctacaaagagatcggcctgctgacctgtgaagccaccgtgaatggccacctgtacaagaccaactacctgacacacagacagaccaacaccatcatcgacgtggtgctgagccctagccacggcattgaactgtctgtgggcgagaagctggtgctgaactgtaccgccagaaccgagctgaacgtgggcatcgacttcaactgggagtaccccagcagcaagcaccagcacaagaaactggtcaaccgggacctgaaaacccagagcggcagcgagatgaagaaattcctgagcaccctgaccatcgacggcgtgaccagaagtgaccagggcctgtacacatgtgccgccagctctggcctgatgaccaagaaaaacagcaccttcgtgcgggtgcacgagaaggacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaatagcacctacagagtggtgtccgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgacaaagaaccaggtgtccctgacctgcctggtcaagggcttctacccttccgatatcgccgtggaatgggagagcaatggccagcctgagaacaactacaagacaacccctcctgtgctggacagcgacggctcattcttcctgtacagcaagctgacagtggacaagagcagatggcagcagggcaacgtgttcagctgctccgtgatgcacgaggccctgcacaaccactacacccagaagtccctgagcctgtctcctggcaaatgagccacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 7 - DNA, 1962 bp, synthetic construct
  misc_feature 1..1962: note = codon optimized REP1 sequence
  source 1..1962: mol_type = other DNA
atggctgatacactgccttctgagtttgatgtgatcgtgattggaactggactgcctgagagtattattgctgctgcttgtagtagaagcggccggagagtgctgcacgtggacagcagatcctactatggcggcaactgggcctctttcagcttttccggcctgctgagctggctgaaggagtaccaggagaactccgacatcgtgtctgatagccccgtgtggcaggaccagatcctggagaatgaggaggccatcgccctgtccaggaaggataagaccatccagcacgtggaggtgttctgctatgccagccaggacctgcacgaggatgtggaggaggcaggcgccctgcagaagaaccacgccctggtgacctccgccaattctacagaggccgccgactccgcctttctgcctaccgaggatgagtccctgtctacaatgtcttgtgagatgctgaccgagcagacacctagctccgatccagagaacgccctggaggtcaatggcgccgaggtgaccggcgagaaggagaaccactgcgacgataagacctgcgtgccaagcacatccgccgaggacatgtccgagaacgtgcctatcgccgaggataccacagagcagccaaagaagaatcgcatcacatacagccagatcatcaaggagggcaggcgcttcaatatcgacctggtgtctaagctgctgtacagccggggcctgctgatcgatctgctgatcaagagcaacgtgtcccgctatgccgagttcaagaatatcaccagaatcctggcctttcgggagggaagagtggagcaggtgccctgcagcagagccgacgtgttcaactccaagcagctgacaatggtggagaagaggatgctgatgaagttcctgacattttgtatggagtacgagaagtatccagatgagtacaagggctatgaggagatcaccttttacgagtatctgaagacccagaagctgacacccaatctgcagtacatcgtgatgcactccatcgccatgacctctgagacagcctctagcaccatcgacggcctgaaggccacaaagaacttcctgcactgcctgggccggtacggcaatacacccttcctgtttcctctgtatggccagggcgagctgccccagtgcttctgtagaatgtgcgccgtgtttggcggcatctattgcctgaggcactctgtgcagtgtctggtggtggacaaggagagccgcaagtgtaaggccatcatcgatcagtttggccagcggatcatctctgagcacttcctggtggaggacagctactttcctgagaacatgtgctccagggtgcagtatcgccagatcagccgggccgtgctgatcaccgatagatccgtgctgaagacagacagcgatcagcagatcagcatcctgaccgtgccagcagaggagccaggcaccttcgccgtgagagtgatcgagctgtgctcctctaccatgacatgtatgaagggcacctacctggtgcacctgacctgcacaagctccaagacagcccgcgaggacctggagagcgtggtgcagaagctgttcgtgccctacaccgagatggagatcgagaacgagcaggtggagaagcctagaatcctgtgggccctgtacttcaacatgagagactctagcgatatctctaggagctgttacaacgatctgccctctaacgtgtacgtgtgcagcggacctgactgtggcctgggaaacgataatgccgtgaagcaggccgagacactgttccaggagatttgccctaacgaggacttttgtccccctccacccaatccagaggatatcatcctggacggcgattccctgcagccagaggcctctgagtcctctgccatccccgaggccaatagcgaaacattcaaagaaagcacaaatctgggaaacctggaagaaagtagtgagtaa

SEQ ID NO: 8 - DNA, 3459 bp, synthetic construct
  misc_feature 1..3459: note = codon optimized RPGR1 sequence
  source 1..3459: mol_type = other DNA
atgagagaacccgaggaactgatgcccgactctggcgccgtgtttaccttcggcaagagcaagttcgccgagaacaaccccggcaagttctggttcaagaacgacgtgccagtgcacctgagctgcggagatgaacactctgccgtggtcaccggcaacaacaagctgtacatgttcggcagcaacaactggggccagctcggcctgggatctaagtctgccatcagcaagcctacctgcgtgaaggccctgaagcctgagaaagtgaaactggccgcctgcggcagaaatcacaccctggtttctaccgaaggcggcaatgtgtatgccaccggcggaaacaatgagggacagcttggactgggcgacaccgaggaaagaaacaccttccacgtgatcagctttttcaccagcgagcacaagatcaagcagctgagcgccggctctaatacctctgccgctctgacagaggacggcagactgtttatgtggggcgacaattctgagggccagatcggactgaagaacgtgtccaatgtgtgcgtgccccagcaagtgacaatcggcaagcctgtgtcttggatcagctgcggctactaccacagcgcctttgtgacaaccgatggcgagctgtatgtgttcggcgagccagagaatggcaagctgggactgcctaaccagctgctgggcaatcacagaacccctcagctggtgtctgagatccccgaaaaagtgatccaggtggcctgtggcggagagcacacagtggtgctgacagagaatgccgtgtacacctttggcctgggccagtttggacaactcggactgggaaccttcctgttcgagacaagcgagcccaaagtgatcgagaacatccgggaccagaccatcagctacatcagctgtggcgagaaccacacagccctgatcacagacatcggcctgatgtacacattcggcgacggaaggcatggaaagctcggacttggcctggaaaacttcaccaaccacttcatccctacgctgtgcagcaacttcctgcggttcattgtgaagctggtggcctgcggaggatgccacatggtggtttttgctgcccctcacagaggcgtggccaaagagattgagttcgacgagatcaacgatacctgcctgagcgtggccaccttcctgccttacagcagcctgacatctggcaacgtgctgcagaggacactgagcgccagaatgcgcagacgggaaagagagagaagccccgacagcttcagcatgagaagaaccctgcctccaatcgagggcacactgggcctgtctgcctgctttctgcctaacagcgtgttccccagatgcagcgagagaaacctgcaagagagcgtgctgagcgagcaggatctgatgcagcctgaggaacccgactacctgctggacgagatgaccaaagaggccgagatcgacaacagcagcacagtggaaagcctgggcgagacaaccgacatcctgaacatgacccacatcatgagcctgaacagcaacgagaagtctctgaagctgagccccgtgcagaagcagaagaagcagcagaccatcggcgagctgacacaggatactgccctgaccgagaacgacgacagcgacgagtacgaagagatgagcgagatgaaggaaggcaaggcctgcaagcagcacgtgtcccagggcatctttatgacccagcctgccaccaccatcgaggccttttccgacgaggaagtggaaatccccgaggaaaaagagggcgccgaggacagcaaaggcaacggcattgaggaacaagaggtggaagccaacgaagagaacgtgaaggtgcacggcggacggaaagaaaagaccgagatcctgagcgacgacctgaccgataaggccgaggtttccgagggcaaagccaagtctgtgggagaagccgaggatggacctgaaggccgcggagatggaacctgtgaagaaggatctagcggagccgagcactggcaggatgaggaacgcgagaagggcgagaaagacaaaggcagaggcgagatggaaagacccggcgagggcgaaaaagagctggccgagaaagaggaatggaagaaacgcgacggcgaagaacaagagcagaaagaaagagagcagggccaccagaaagaacggaatcaagagatggaagaaggcggcgaggaagaacacggcgaaggggaagaagaggaaggcgaccgagaggaagaagaagagaaagaaggcgaaggcaaagaagaaggcgagggcgaagaggtggaaggcgagcgtgaaaaagaagagggcgaacgcaagaaagaagaacgcgccggaaaagaggaaaaaggcgaggaagagggcgaccaaggcgaaggcgaggaagaagaaactgaaggcagaggggaagagaaagaggaaggcggcgaagtcgaaggcggagaggttgaagaaggcaaaggcgagcgagaagaggaagaagaagaaggcgaaggcgaggaagaggaaggcgaaggcgaagaggaagaaggcgaaggggaagaagaagaaggcgaaggcaagggcgaagaggagggcgaagaaggcgagggcgaagaggagggcgaagaaggcgaaggcgagggcgaagaagaagaaggcgaaggcgaaggcgaggaagaaggcgaaggcgaaggggaagaagaggaaggcgaaggcgaaggcgaagaagaaggcgaaggcgagggcgaagaggaagaaggcgaaggcaaaggggaagaagaaggcgaggaaggcgaaggcgaaggcgaggaagaagaaggcgaaggcgagggcgaagatggcgaaggcgaaggcgaagaggaagagggcgagtgggagggcgaagaagaggaaggcgaaggcgagggcgaagaggaaggcgaaggcgagggcgaagaaggcgaaggcgaaggcgaggaagaggaaggcgaaggcgaaggggaagaagaagagggcgaagaagaaggcgaagaggaaggcgaaggggaagaagaaggcgaaggcgaaggcgaagaagaggaagagggcgaagttgaaggcgaggttgagggcgaagaaggcgaaggcgaaggggaagaagaagaaggcgaggaagaaggggaagagagagaaaaagaaggcgagggcgaagaaaaccgccggaaccgcgaagaggaagaggaagaagagggcaagtaccaagagactggcgaggaagagaacgagcggcaggatggcgaagagtacaagaaggtgtccaagatcaagggcagcgtgaagtacggcaagcacaagacctaccagaagaagtccgtcaccaacacgcaaggcaatggaaaagaacagcggagcaagatgcccgtgcagtccaagaggctgctgaagaatggccctagcggcagcaagaaattctggaacaatgtgctgccccactacctcgagctgaagtga

SEQ ID NO: 9 - protein, 701 aa, synthetic construct
  REGION 1..701: note = synthetic polypeptide
MRLLAKIICLMLWAICVAEDCNELPPRRNTEILTGSWSDQTYPEGTQAIYKCRPGYRSLGNIIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 10 - DNA, 4432 bp, synthetic construct
  misc_feature 1..4432: note = synthetic polynuclelotide
  source 1..4432: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaattccccggggatccactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacctgcaggtggatatcttgctagcacgccaccatgcgcctgctggccaagatcatctgcctgatgctgtgggccatctgcgtggccgaggactgtaacgagctgccccctcggagaaatacagagatcctgaccggctcttggagcgatcagacctacccagagggcacacaggccatctacaagtgcaggcccggctatcgctccctgggcaatatcatcatggtgtgcaggaagggagagtgggtggccctgaacccactgagaaagtgccagaagaggccatgtggacacccaggcgacacacctttcggcacctttacactgaccggcggcaacgtgttcgagtacggcgtgaaggccgtgtatacctgcaatgagggctaccagctgctgggcgagatcaactaccgggagtgtgacacagatggctggaccaatgatatccccatctgcgaggtggtgaagtgtctgccagtgaccgcccccgagaacggcaagatcgtgagctccgccatggagcctgacagagagtatcacttcggccaggccgtgaggttcgtgtgcaatagcggctacaagatcgagggcgatgaggagatgcactgttccgacgatggcttctggtctaaggagaagcccaagtgcgtggagatctcctgtaagtctcctgacgtgattaacggcagcccaatctcccagaagatcatctataaggagaatgagcggtttcagtacaagtgcaacatgggctacgagtattccgagagaggcgatgccgtgtgcacagagtctggatggaggcccctgcctagctgcgaggagaagtccaccctgaagccttgtgactatccagatatcaagcacggcggcctgtatcacgagaatatgaggcgcccttacttcccagtggccgtgggcaagtactattcctactattgcgacgagcactttgagacaccatccggctcttactgggaccacatccactgtacccaggatggctggtctcccgccgtgccttgcctgagaaagtgttacttcccctatctggagaacggctacaaccagaattatggcaggaagtttgtgcagggcaagtccatcgacgtggcatgccacccaggatacgcactgcctaaggcacagaccacagtgacctgtatggagaatggctggagcccaacacccaggtgcatccgcgtgaagacctgttccaagagcagcatcgacatcgagaacggcttcatcagcgagtcccagtacacctatgccctgaaggagaaggccaagtatcagtgcaagctgggatacgtgaccgcagacggagaaacatctggcagcatcacctgcggcaaggatggctggtctgcccagcccacatgcatcaagagcatcaagaccgactgtctgtctctgcccagctttgagaatgccatccctatgggcgagaagaaggacgtgtacaaggccggcgagcaggtgacatacacctgcgccacatactataagatggacggcgccagcaacgtgacctgtatcaattcccggtggacaggcagacctacctgcagggatacaagctgcgtgaacccacccaccgtgcagaatgcctatatcgtgtcccgccagatgtctaagtaccctagcggcgagcgggtgagatatcagtgccggtccccatacgagatgttcggcgacgaggaagtgatgtgcctgaacggcaattggacagagcctccacagtgcaaggatagcaccggcaagtgtggaccacctccaccaatcgacaacggcgatatcacatcctttccactgagcgtgtacgcccccgcctcctctgtggagtatcagtgccagaacctgtaccagctggagggcaataagcgcatcacctgtcggaacggccagtggtctgagcctccaaagtgcctgcacccttgcgtgatctccagagagatcatggagaactataatatcgccctgcgctggacagccaagcagaagctgtactctcggaccggcgagagcgtggagttcgtgtgcaagcggggctatagactgagctcccgcagccacacactgcggaccacatgctgggacggcaagctggagtacccaacctgtgccaagaggtgagccacgcgtaacacgtgcatgcgagagatctgcggccgcgagctcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatcctagccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 11 - DNA, 4696 bp, synthetic construct
  misc_feature 1..4696: note = synthetic polynucleotide
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgcgcctgctggccaagatcatctgcctgatgctgtgggccatctgcgtggccgaggactgtaacgagctgccccctcggagaaatacagagatcctgaccggctcttggagcgatcagacctacccagagggcacacaggccatctacaagtgcaggcccggctatcgctccctgggcaatatcatcatggtgtgcaggaagggagagtgggtggccctgaacccactgagaaagtgccagaagaggccatgtggacacccaggcgacacacctttcggcacctttacactgaccggcggcaacgtgttcgagtacggcgtgaaggccgtgtatacctgcaatgagggctaccagctgctgggcgagatcaactaccgggagtgtgacacagatggctggaccaatgatatccccatctgcgaggtggtgaagtgtctgccagtgaccgcccccgagaacggcaagatcgtgagctccgccatggagcctgacagagagtatcacttcggccaggccgtgaggttcgtgtgcaatagcggctacaagatcgagggcgatgaggagatgcactgttccgacgatggcttctggtctaaggagaagcccaagtgcgtggagatctcctgtaagtctcctgacgtgattaacggcagcccaatctcccagaagatcatctataaggagaatgagcggtttcagtacaagtgcaacatgggctacgagtattccgagagaggcgatgccgtgtgcacagagtctggatggaggcccctgcctagctgcgaggagaagtccaccctgaagccttgtgactatccagatatcaagcacggcggcctgtatcacgagaatatgaggcgcccttacttcccagtggccgtgggcaagtactattcctactattgcgacgagcactttgagacaccatccggctcttactgggaccacatccactgtacccaggatggctggtctcccgccgtgccttgcctgagaaagtgttacttcccctatctggagaacggctacaaccagaattatggcaggaagtttgtgcagggcaagtccatcgacgtggcatgccacccaggatacgcactgcctaaggcacagaccacagtgacctgtatggagaatggctggagcccaacacccaggtgcatccgcgtgaagacctgttccaagagcagcatcgacatcgagaacggcttcatcagcgagtcccagtacacctatgccctgaaggagaaggccaagtatcagtgcaagctgggatacgtgaccgcagacggagaaacatctggcagcatcacctgcggcaaggatggctggtctgcccagcccacatgcatcaagagcatcaagaccgactgtctgtctctgcccagctttgagaatgccatccctatgggcgagaagaaggacgtgtacaaggccggcgagcaggtgacatacacctgcgccacatactataagatggacggcgccagcaacgtgacctgtatcaattcccggtggacaggcagacctacctgcagggatacaagctgcgtgaacccacccaccgtgcagaatgcctatatcgtgtcccgccagatgtctaagtaccctagcggcgagcgggtgagatatcagtgccggtccccatacgagatgttcggcgacgaggaagtgatgtgcctgaacggcaattggacagagcctccacagtgcaaggatagcaccggcaagtgtggaccacctccaccaatcgacaacggcgatatcacatcctttccactgagcgtgtacgcccccgcctcctctgtggagtatcagtgccagaacctgtaccagctggagggcaataagcgcatcacctgtcggaacggccagtggtctgagcctccaaagtgcctgcacccttgcgtgatctccagagagatcatggagaactataatatcgccctgcgctggacagccaagcagaagctgtactctcggaccggcgagagcgtggagttcgtgtgcaagcggggctatagactgagctcccgcagccacacactgcggaccacatgctgggacggcaagctggagtacccaacctgtgccaagaggtgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 12 - DNA, 4696 bp, synthetic construct
  misc_feature 1..4696: note = synthetic polynucleotide
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgaggctgctggccaagatcatctgcctgatgctgtgggccatctgtgtggctgaggactgtaatgagctgccccctaggagaaatacagagatcctgactggctcttggtctgatcagacctacccagagggcacacaggccatctacaagtgcaggcctggctatagatccctgggcaatatcatcatggtgtgcaggaagggagagtgggtggccctgaacccactgagaaagtgccagaagaggccatgtggacacccaggagacacaccttttggcacctttacactgacagggggcaatgtgtttgagtatggtgtgaaggcagtgtatacctgcaatgagggctaccagctgctgggagagatcaactacagggagtgtgacacagatggctggaccaatgatatccccatctgtgaggtggtgaagtgtctgccagtgactgcccctgagaatggcaagatagtgagctctgccatggagcctgacagagagtatcactttggccaggctgtgaggtttgtgtgcaatagtggctacaagattgagggggatgaggagatgcactgttcagatgatggcttctggtctaaggagaagcccaagtgtgtggagatctcctgtaagtctcctgatgtgattaatggcagcccaatctcccagaagatcatctataaggagaatgagagatttcagtacaagtgcaacatgggctatgagtattcagagagaggtgatgctgtgtgcacagagtctggatggaggcccctgcctagctgtgaggagaagtccaccctgaagccttgtgactatccagatatcaagcatggaggcctgtatcatgagaatatgaggaggccttacttcccagtggctgtgggcaagtactattcctactattgtgatgagcactttgagacaccatctggctcttactgggaccacatccactgtacccaggatggctggtctcctgctgtgccttgcctgagaaagtgttacttcccctatctggagaatggctacaaccagaattatggcaggaagtttgtgcagggcaagtccattgatgtggcatgccacccaggatatgcactgcctaaggcacagaccacagtgacctgtatggagaatggctggagcccaacacccaggtgcatcagggtgaagacctgttccaagagcagcattgacatagagaatggcttcatctcagagtcccagtacacctatgccctgaaggagaaggccaagtatcagtgcaagctgggatatgtgacagcagatggagaaacatctggcagcatcacctgtggcaaggatggctggtctgcccagcccacatgcatcaagagcatcaagactgactgtctgtctctgcccagctttgagaatgccatccctatgggtgagaagaaggatgtgtacaaggctggagagcaggtgacatacacctgtgccacatactataagatggatggagccagcaatgtgacctgtatcaattccagatggacaggcagacctacctgcagggatacaagctgtgtgaacccacccactgtgcagaatgcctatattgtgtccaggcagatgtctaagtacccttctggagagagagtgagatatcagtgcagatccccatatgagatgtttggggatgaggaagtgatgtgcctgaatggcaattggacagagcctccacagtgcaaggatagcactggcaagtgtggaccacctccaccaattgacaatggagatatcacatcctttccactgtcagtgtatgcccctgcctcctctgtggagtatcagtgccagaacctgtaccagctggagggcaataagagaatcacctgtagaaatggccagtggtctgagcctccaaagtgcctgcacccttgtgtgatctccagagagatcatggagaactataatatagccctgaggtggacagccaagcagaagctgtactctaggactggggagtcagtggagtttgtgtgcaagagaggctatagactgagctccagaagccacacactgaggaccacatgctgggatggcaagctggagtacccaacctgtgccaagaggtgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 13 - DNA, 4696 bp, synthetic construct
  misc_feature 1..4696: note = synthetic polynucleotide
  source 1..4696: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctatcgattgaaccaggcgcgcccagtttaaacagtccggaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagtctagagtcgacggtcaccggtcagctagcacgccaccatgagacttctagcaaagattatttgccttatgttatgggctatttgtgtagcagaagattgcaatgaacttcctccaagaagaaatacagaaattctgacaggttcctggtctgaccaaacatatccagaaggcacccaggctatctataaatgcaggcctggatatagatctcttggaaatatcataatggtatgcaggaagggagaatgggttgctcttaatccattaaggaaatgtcagaaaaggccctgtggacatcctggagatactccttttggtacttttacccttacaggaggaaatgtgtttgaatatggtgtaaaagctgtgtatacatgtaatgaggggtatcaattgctaggtgagattaattacagggaatgtgacacagatggatggaccaatgatattcctatatgtgaagttgtgaagtgtttaccagtgacagcaccagagaatggaaaaattgtcagtagtgcaatggaaccagatagagaataccattttggacaagcagtaagatttgtatgtaactcaggctacaagattgaaggagatgaagaaatgcattgttcagatgatggtttttggagtaaagagaaaccaaagtgtgtggaaatttcatgcaaatccccagatgttataaatggatctcctatatctcagaagattatttataaggagaatgaaagatttcaatataaatgtaacatgggttatgaatacagtgaaagaggagatgctgtatgcactgaatctggatggagacctttgccttcatgtgaagaaaaatcaaccttgaaaccttgtgattatccagacattaaacatggaggtctatatcatgagaatatgagaagaccatactttccagtagctgtaggaaaatattactcctattactgtgatgaacattttgagactccttcaggaagttactgggatcacattcattgcacacaagatggatggagtccagcagtaccatgcctcagaaaatgttattttccttatttggaaaatggatataatcaaaattatggaagaaagtttgtacagggtaaatctatagatgttgcctgccatcctggctatgctcttccaaaagctcagaccacagttacatgtatggagaatggctggtctcctactcccagatgcatcagagtcaaaacatgttccaaatcaagtatagatattgagaatgggtttatttctgaatctcagtatacatatgccttaaaagaaaaagctaaatatcaatgcaaactaggatatgtaacagcagatggtgaaacatcaggatcaattacatgtgggaaagatggatggtcagctcaacccacctgcattaaatctataaaaacagattgtctcagtttacctagctttgaaaatgccatacccatgggagagaagaaggatgtgtataaggctggtgagcaagtgacttacacttgtgcaacatattacaaaatggatggagccagtaatgtaacatgcattaatagcagatggacaggaaggccaacatgcagagacacctcctgtgtgaatcctcccacagtacaaaatgcttatatagtgagtagacagatgagtaaatatccatctggtgagagagtaagatatcaatgtaggagcccttatgaaatgtttggggatgaagaagtgatgtgtttaaatggaaactggacagaaccacctcaatgcaaagattctacaggaaaatgtgggccccctccacctattgacaatggggacattacttcattccccttgtcagtatatgctccagcttcatcagttgagtaccaatgccagaacttgtatcaacttgagggtaacaagaggataacatgtagaaatggacaatggtcagaaccaccaaaatgcttacatccctgtgtaatatccagggaaattatggaaaattataacatagcattaaggtggacagccaaacagaagctttattctagaacaggtgaatcagttgaatttgtgtgtaaaagaggatatagactttcatcaaggtctcacacattgaggacaacatgttgggatgggaaactggagtatccaacttgtgcaaaaagatgagcctaggactgtacagtacgcgtaacacgtgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcaatgcatggtcccagccttcattgagcctattggtcccccctcagaacccacccacctggcagtagaggatgtctcttgagacaccactgtctccctcaagttcaggaggcccccattaattaagagagagtgggagcaggaggcctgtcagatggctactcttaagtggagtactgcccagagggctgctcagagtgggtggctgtaaccctgcaggggctgacagagcacacatccatactggtgaaggacctgtgacccactggccagactggccggccggaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 14 - protein, 735 aa, Adeno-associated virus 2 [sequence omitted]

(19) United States
(12) Patent Application Publication
(10) Pub. No.: US 2026/0295077 A1
(43) Pub. Date: Oct. 1, 2026

(54) Adeno-associated Virus Formulations

(71) Applicant: 4D MOLECULAR THERAPEUTICS INC., Emeryville, CA (US)

(72) Inventors: CATHERINE T. BARGLOW, Emeryville, CA (US); JOHNNY C. GONZALES, Emeryville, CA (US)

(73) Assignee: 4D MOLECULAR THERAPEUTICS INC., Emeryville, CA (US)

(21) Appl. No.: 19/490,178

(22) PCT Filed: Jun. 4, 2024
(86) PCT No.: PCT/US2024/032381

§ 371 Date: Dec. 4, 2025

Related U.S. Application Data
(60) Provisional application No. 63/507,266, filed on Jun. 9, 2023.

(51) Int. Cl. A61K 48/00; A61K 38/17; A61K 47/02; A61K 47/34

(52) U.S. Cl. CPC A61K 48/0033; A61K 38/1725; A61K 47/02; A61K 47/34; A61K 48/005

(57) ABSTRACT

Pharmaceutical compositions comprising adeno-associated virus (AAV) are provided herein. In some aspects, the pharmaceutical compositions comprise about 5 mM to about 20 mM Tris buffer, about 100 mM to about 250 mM sodium chloride, about 0.001% (w/v) to about 0.01% (w/v) Pluronic F68, and has a pH between 7.0 and 9.0. Methods of preparing a pharmaceutical composition comprising AAV, methods of treating a VEGF-associated disorder in a subject, and methods of storing AAV compositions are also provided.

### CROSS REFERENCE TO RELATED APPLICATIONS

This application claims the benefit of U.S. Provisional Patent Application Ser. No. 63/507,266, filed Jun. 9, 2023, the entire contents of which are incorporated herein by reference.

### SEQUENCE LISTING SUBMISSION VIA EFS-WEB

A computer readable XML file, entitled “090400-5024 WO Sequence Listing” created on Jun. 3, 2024, with a file size of about 49,743 bytes contains the sequence listing for this application and is hereby incorporated by reference in its entirety.

### BACKGROUND OF THE INVENTION

It is understood that AAV formulations intended for human administration should be not only safe, sterile, and of good manufacturing practice (GMP) grade, these formulations should also exhibit and promote the long-term stability of the AAV, minimizing loss of AAV potency during the manufacture, packaging, and storage processes. Though the efforts to design such AAV formulations have been great, there still remains a need for improved AAV formulation.

### SUMMARY OF THE INVENTION

Described herein are formulations compatible for human administration which address the unmet needs described above. Advantageously, in some embodiments, the formulations are suitable for long-term storage of AAV, minimizing loss of AAV potency, and advantageously prevent subvisible particle formation and prevent adsorption to the surfaces of the containers in which the AAV are packaged and stored and of the machinery used during manufacture.

In some aspects, the pharmaceutical composition of the present disclosure comprises: adeno-associated virus (AAV) and about 5 mM to about 20 mM of a buffering agent, about 100 mM to about 250 mM of a pharmaceutically acceptable salt, about 0.0001% (w/v) to about 0.01% (w/v) of a non-ionic surfactant and preferably has a pH of about 7.0 to about 9.0.

In some aspects, the pharmaceutical composition of the present disclosure comprises: AAV (e.g., AAV2 or a variant thereof), about 5 mM to about 20 mM Tris buffer, about 100 mM to about 250 mM sodium chloride, and about 0.0001% (w/v) to about 0.01% (w/v) Pluronic F68, and preferably has a pH of about 7.0 to about 9.0.

In some embodiments, the invention provides a liquid formulation. In certain embodiments, the formulation is lyophilized from a liquid formulation. In certain aspects, the lyophilized pharmaceutical composition of the present disclosure is lyophilized from a liquid formulation.

In other embodiments, methods of preparing a pharmaceutical composition comprising AAV are provided. In certain aspects, the method comprises combining about 5 mM to about 20 mM Tris buffer, about 100 mM to about 250 mM sodium chloride, about 0.0001% (w/v) to about 0.01% (w/v) Pluronic F68, and AAV, and adjusting the pH to a pH of between about 7.0 to about 9.0, thereby obtaining a pharmaceutical composition comprising AAV. Methods of storing a composition comprising AAV are also provided.

Methods for delivering a heterologous nucleic acid comprising a nucleotide sequence encoding a gene product to a mammalian subject are provided. In exemplary aspects, the method comprises administering to the mammal a pharmaceutical composition as herein described. In some aspects, the heterologous nucleic acid is delivered to a retinal cell of the subject, e.g., a photoreceptor cell (e.g., rods; cones), a retinal ganglion cell (RGC), a glial cell (e.g., a Müller glial cell, a microglial cell), a bipolar cell, an amacrine cell, a horizontal cell, and/or a retinal pigmented epithelium (RPE) cell of the subject.

Methods of treating a subject for a disorder treatable by gene therapy are provided by the present disclosure. In exemplary aspects, the method comprises administering to the subject a pharmaceutical composition as described herein in an amount effective to treat the disorder. In some aspects, the disorder is an ocular disorder. In particularly preferred embodiments, the disorder is a VEGF-associated ocular disease such as wet or dry age-related macular degeneration (AMD) or geographic atrophy secondary to AMD.

In certain embodiments, the pharmaceutical composition of the present disclosure comprises: AAV and about 8-12 mM Tris buffer, about 160-200 mM sodium chloride, about 0.0005%-0.01% (w/v) Pluronic F68 and preferably comprises a pH of about 7.4 to 8.1. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 7.6. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 7.7. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 7.8. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 7.9. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 8.0. In some embodiments, the invention provides a liquid formulation. In certain embodiments, the formulation is lyophilized from a liquid formulation.

In certain embodiments, the pharmaceutical composition of the present disclosure comprises: AAV and about 8-12 mM, preferably about 10 mM, Tris buffer, about 160-200 mM, preferably about 180 mM, sodium chloride, about 0.0005%-0.01% (w/v) Pluronic F68 and comprises a pH of about 8.1. In some embodiments, the invention provides a liquid formulation. In certain embodiments, the formulation is lyophilized from a liquid formulation.

In some embodiments, the pharmaceutical composition of the present disclosure comprises: AAV and about 9 mM to about 20 mM buffering agent; about 140 to about 200 mM pharmaceutically acceptable salt; about 0.001% (w/v) to about 0.01% (w/v) non-ionic surfactant; and a pH of about 7.3 to 8.6. In certain embodiments, the pharmaceutical composition of the present disclosure comprises: AAV and about 9-20 mM Tris buffer, about 140-200 mM sodium chloride, about 0.001%-0.01% (w/v) Pluronic F68 and comprises a pH of about 7.3 to 8.6. In some embodiments, the invention provides a liquid formulation. In certain embodiments, the formulation is lyophilized from a liquid formulation.

In certain embodiments, the pharmaceutical composition does not comprise a divalent cation and/or does not comprise a sugar or sugar alcohol.

Generally, the AAV formulations provided herein are suitable for pharmaceutical administration. In certain embodiments, the AAV is AAV1, AAV2, AAV3, AAV4, AAV5, AAV6, AAV7, AAV8, AAV9, or AAV10. In certain embodiments, the AAV is AAV 2, AAV 5, AAV 8, or AAV 9. In certain embodiments, the AAV is AAV2 or a variant of AAV2.

In some preferred embodiments, the AAV is a recombinant AAV (rAAV) comprising a heterologous nucleic acid encoding a gene product and a capsid protein comprising a peptide insertion of from about 7 amino acids to about 20 amino acids (a “heterologous peptide” or “peptide insertion”) in the GH-loop of the capsid protein, preferably in a surface-exposed region of the GH-loop, relative to a corresponding parental AAV capsid protein. In certain embodiments, the peptide is inserted following any of the amino acids in positions 584-591 in VP1 of AAV2 or a corresponding position in another AAV serotype (i.e., the insertion site is between amino acids 587 and 588 of VP1 of AAV2 or is between amino acids 588 and 589, between amino acids 584 and 585, between amino acids 585 and 586, between amino acids 586 and 587, between amino acids 590 and 591, or is between amino acids 591 and 592 of AAV2 or the corresponding positions in the capsid protein of another AAV serotype). In certain preferred embodiments, the insertion site is between amino acids 587 and 588 of VP1 of AAV2 or is between amino acids 588 and 589 of AAV2 or the corresponding positions in the capsid protein of another AAV serotype. In some embodiments, the capsid protein further comprises one or more amino acid substitutions relative to VP1 capsid of AAV2 or one or more corresponding substitutions in another AAV serotype, preferably wherein the capsid protein further comprises a P34A amino acid substitution relative to VP1 capsid of AAV2 or the corresponding substitution in another AAV serotype.

### DESCRIPTION OF THE DRAWINGS

FIG. 1 Representative background membrane images for 3-day storage at 37° C.

FIG. 2 Aflibercept expression, from pH 6.0-7.0, after 2-week storage at 25° C./60% relative humidity (RH).

FIG. 3 Aflibercept expression, from pH 7.0-7.8, after 2-week storage at 25° C./60% relative humidity (RH).

FIG. 4 illustrates colloidal stability (>90% Area (10-100 d·nm) by Intensity) of the tested formulations across a range of sodium chloride concentrations

FIG. 5 illustrates Final Fluorescence values by Differential Scanning Fluorimetry (DSF) at the specified sodium chloride concentrations following 0.45 μm filtration.

FIG. 6 illustrates the percentage recovery after 3 Freeze-Thaw (3×FT) by DSF at the specified sodium chloride concentrations.

### DETAILED DESCRIPTION OF THE INVENTION

### Definitions

As used herein, the term “AAV” refers to adeno-associated virus in both naturally occurring and recombinant forms (rAAV), and encompasses mutant forms of AAV. The term AAV further includes, but is not limited to, AAV type 1, AAV type 2, AAV type 3, AAV type 4, AAV type 5, AAV type 6, AAV type 7, AAV type 8, AAV type 9, AAV type 10, avian AAV, bovine AAV, canine AAV, equine AAV, ovine AAV, primate AAV, and non-primate AAV. In certain embodiments, the AAV is AAV2 or a variant thereof.

A “pharmaceutically acceptable salt” is a salt that can be formulated into a compound or conjugate for pharmaceutical use including, e.g., metal salts (sodium, potassium, magnesium, calcium, etc.) and salts of ammonia or organic amines that is safe for administration to a subject (e.g., a human) in a drug formulation (see, for example, Berge, et al. “Pharmaceutical Salts,” J. Pharm. Sci. 1977; 66:1, which is incorporated herein by reference in its entirety and for all purposes.). Suitable “pharmaceutically acceptable salts” include, but are not limited to, metal salts such as sodium, potassium and cesium salts; alkaline earth metal salts such as calcium and magnesium salts; organic amine salts such as triethylamine, guanidine and N-substituted guanidine salts, acetamidine and N-substituted acetamidine, pyridine, picoline, ethanolamine, triethanolamine, dicyclohexylamine, and N,N′-dibenzylethylenediamine salts. “Pharmaceutically acceptable salts” (of basic nitrogen centers) include, but are not limited to inorganic acid salts such as the hydrochloride, hydrobromide, sulfate, phosphate; organic acid salts such as trifluoroacetate and maleate salts; sulfonates such as methanesulfonate, ethanesulfonate, benzenesulfonate, p-toluenesulfonate, camphor sulfonate and naphthalenesulfonate; amino acid salts such as arginate, alaninate, asparginate and glutamate; and carbohydrate salts such as gluconate and galacturonate. The selection and use of pharmaceutically acceptable salts is well known in the art, for example, see Stahl and Wermuth, Pharmaceutical Salts: Properties, Selection, and Use, 2nd Revised edition, Wiley, Hoboken, N.J., which is incorporated herein by reference in its entirety and for all purposes. Non-limiting examples of pharmaceutically acceptable salts include, without limitation, sodium salts, ammonium salts, potassium salts (e.g, sodium, ammonium, and potassium chloride; sodium, ammonium, and potassium acetate; sodium, ammonium, and potassium citrate; sodium, ammonium, and potassium phosphate; sodium, ammonium, and potassium fluoride; sodium, ammonium, and potassium bromide; and sodium, ammonium, and potassium iodide).

The term “isolated” designates a biological material (cell, nucleic acid or protein) that has been removed from its original environment (the environment in which it is naturally present). For example, a polynucleotide present in the natural state in a plant or an animal is not isolated, however the same polynucleotide separated from the adjacent nucleic acids in which it is naturally present, is considered “isolated.”

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions can be present in a single polynucleotide construct, e.g., on a single vector, or in separate polynucleotide constructs, e.g., on separate (different) vectors. It follows, then that a single vector can contain just a single coding region, or comprise two or more coding regions.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites and stem-loop structures. If a coding region is intended for expression in a eukaryotic cell, a polyadenylation signal and transcription termination sequence will usually be located 3′ to the coding sequence.

As used herein, the term “nucleic acid” is interchangeable with “polynucleotide” or “nucleic acid molecule” and a polymer of nucleotides is intended.

A polynucleotide which encodes a gene product, e.g., a polypeptide, an interfering RNA such as a primary miRNA, short hairpin RNA (shRNA), or a small interfering RNA (siRNA), can include a promoter and/or other transcription or translation control elements operably associated with one or more coding regions. In an operable association a coding region for a gene product, e.g., a polypeptide, is associated with one or more regulatory regions in such a way as to place expression of the gene product under the influence or control of the regulatory region(s). For example, a coding region and a promoter are “operably associated” if induction of promoter function results in the transcription of mRNA encoding the gene product encoded by the coding region, and if the nature of the linkage between the promoter and the coding region does not interfere with the ability of the promoter to direct the expression of the gene product or interfere with the ability of the DNA template to be transcribed. Other transcription control elements, besides a promoter, for example enhancers, operators, repressors, and transcription termination signals, can also be operably associated with a coding region to direct gene product expression.

“Transcriptional control sequences” refer to DNA regulatory sequences, such as promoters, enhancers, terminators, and the like, that provide for the expression of a coding sequence in a host cell. A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit beta-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

“Promoter” and “promoter sequence” are used interchangeably and refer to a DNA sequence capable of controlling the expression of a coding sequence or functional RNA. In general, a coding sequence is located 3′ to a promoter sequence. Promoters can be derived in their entirety from a native gene, or be composed of different elements derived from different promoters found in nature, or even comprise synthetic DNA segments. It is understood by those skilled in the art that different promoters can direct the expression of a gene in different tissues or cell types, or at different stages of development, or in response to different environmental or physiological conditions. Promoters that cause a gene to be expressed in most cell types at most times are commonly referred to as “constitutive promoters.” Promoters that cause a gene to be expressed in a specific cell type are commonly referred to as “cell-specific promoters” or “tissue-specific promoters.” Promoters that cause a gene to be expressed at a specific stage of development or cell differentiation are commonly referred to as “developmentally-specific promoters” or “cell differentiation-specific promoters.” Promoters that are induced and cause a gene to be expressed following exposure or treatment of the cell with an agent, biological molecule, chemical, ligand, light, or the like that induces the promoter are commonly referred to as “inducible promoters” or “regulatable promoters.” It is further recognized that since in most cases the exact boundaries of regulatory sequences have not been completely defined, DNA fragments of different lengths can have identical promoter activity.

The term “plasmid” refers to an extra-chromosomal element often carrying a gene that is not part of the central metabolism of the cell, and usually in the form of circular double-stranded DNA molecules. Such elements can be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the world wide web at ncbi.nlm.nih.gov/BLAST/. Another alignment algorithm is FASTA, available in the Genetics Computing Group (GCG) package, from Madison, Wis., USA. Other techniques for alignment are described in Methods in Enzymology, vol. 266: Computer Methods for Macromolecular Sequence Analysis (1996), ed. Doolittle, Academic Press, Inc. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The term “amino acid substitution” and its synonyms described above are intended to encompass modification of an amino acid sequence by replacement of an amino acid with another, substituting, amino acid. The substitution may be a conservative substitution. It may also be a non-conservative substitution. The term conservative, in referring to two amino acids, is intended to mean that the amino acids share a common property recognized by one of skill in the art. For example, amino acids having hydrophobic nonacidic side chains, amino acids having hydrophobic acidic side chains, amino acids having hydrophilic nonacidic side chains, amino acids having hydrophilic acidic side chains, and amino acids having hydrophilic basic side chains. Common properties may also be amino acids having hydrophobic side chains, amino acids having aliphatic hydrophobic side chains, amino acids having aromatic hydrophobic side chains, amino acids with polar neutral side chains, amino acids with electrically charged side chains, amino acids with electrically charged acidic side chains, and amino acids with electrically charged basic side chains. Both naturally occurring and non-naturally occurring amino acids are known in the art and may be used as substituting amino acids in embodiments. Methods for replacing an amino acid are well known to the skilled in the art and include, but are not limited to, mutations of the nucleotide sequence encoding the amino acid sequence. Reference to “one or more” herein is intended to encompass the individual embodiments of, for example, 1, 2, 3, 4, 5, 6, or more.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease (and/or symptoms caused by the disease) from occurring in a subject which may be predisposed to the disease or at risk of acquiring the disease but has not yet been diagnosed as having it; (b) inhibiting the disease (and/or symptoms caused by the disease), i.e., arresting its development; and (c) relieving the disease (and/or symptoms caused by the disease), i.e., causing regression of the disease (and/or symptoms caused by the disease), i.e., ameliorating the disease and/or one or more symptoms of the disease.

The terms “individual,” “host,” “subject,” and “patient” are used interchangeably herein, and refer to a mammal, including, but not limited to, humans; non-human primates, including simians; mammalian sport animals (e.g., horses); mammalian farm animals (e.g., sheep, goats, etc.); mammalian pets (dogs, cats, etc.); and rodents (e.g., mice, rats, etc.).

The term “effective amount” as used herein is an amount sufficient to effect beneficial or desired clinical results. An effective amount can be administered in one or more administrations. For purposes of this disclosure, an effective amount of a compound (e.g., an infectious rAAV virion) is an amount that is sufficient to palliate, ameliorate, stabilize, reverse, prevent, slow or delay the progression of (and/or symptoms associated with) a particular disease state (e.g., a disorder associated with complement dysfunction). Accordingly, an effective amount of an infectious rAAV virion is an amount of the infectious rAAV virion that is able to effectively deliver a heterologous nucleic acid to a target cell (or target cells) of the individual. Effective amounts may be determined preclinically by, e.g., detecting in the cell or tissue the gene product (RNA, protein) that is encoded by the heterologous nucleic acid sequence using techniques that are well understood in the art, e.g. RT-PCR, western blotting, ELISA, fluorescence or other reporter readouts, and the like. Effective amounts may be determined clinically by, e.g. detecting a change in the onset or progression of disease using methods known in the art, e.g. 6-minute walk test, left ventricular ejection fraction, hand-held dynamometry, Vignos Scale and the like as described herein and as known in the art.

### DETAILED DESCRIPTION

The present disclosure provides formulations, e.g., pharmaceutical compositions, compatible for human administration which also are suitable for long-term storage of AAV and minimizing loss of AAV potency. The formulations provided herein are advantageous, because the formulations prevent subvisible particle formation, and retain significant AAV activity when stored for extended periods of time. In certain embodiments, the pharmaceutical compositions provided herein reduce or retard degradation and/or aggregation.

In certain embodiments, the present invention provides formulations of AAV comprising a therapeutically effective amount or dose of an AAV, a pharmaceutically acceptable salt, a non-ionic surfactant, and one or more buffering agents providing a pH as herein described to the formulation. Generally, the AAV formulations provided herein are suitable for pharmaceutical administration. In certain embodiments, the AAV is AAV2 or a variant thereof.

### AAV Formulations and Compositions

In exemplary aspects, the pharmaceutical composition of the present disclosure comprises: adeno-associated virus (AAV) and about 5 mM to about 20 mM of a buffering agent, about 100 mM to about 250 mM of a pharmaceutically acceptable salt, about 0.0001% (w/v) to about 0.01% (w/v) of a non-ionic surfactant, and a pH of at least about 7.0.

In exemplary aspects, the pharmaceutical composition of the present disclosure comprises: adeno-associated virus (AAV) and about 5 mM to about 20 mM of a buffering agent, about 100 mM to about 200 mM of a pharmaceutically acceptable salt, about 0.001% (w/v) to about 0.01% (w/v) of a non-ionic surfactant, and a pH of between about 7.0 and about 9.0.

In exemplary aspects, the pharmaceutical composition of the present disclosure comprises: adeno-associated virus (AAV) and about 5 mM to about 20 mM of a buffering agent, about 150 mM to about 200 mM of a pharmaceutically acceptable salt, about 0.001% (w/v) to about 0.01% (w/v) of a non-ionic surfactant, and a pH of at least about 7.4.

In exemplary aspects, the pharmaceutical composition of the present disclosure comprises: adeno-associated virus (AAV) and about 10 mM to about 20 mM of a buffering agent, about 150 mM to about 200 mM of a pharmaceutically acceptable salt, about 0.001% (w/v) to about 0.01% (w/v) of a non-ionic surfactant, and a pH of at least about 7.4

In certain aspects, the composition is a sterile composition. By “sterile” it is meant that there are substantially no immunogenic components in the composition, such as for example substantially no microbes (e.g., fungi, bacteria, viruses, spore forms, etc.).

In some embodiments, the invention provides a liquid formulation. In certain embodiments, the formulation is lyophilized from a liquid formulation.

In exemplary aspects, the pharmaceutical composition of the present disclosure comprises about 5 mM to about 20 mM, about 5 mM to about 15 mM, about 10 mM to about 20 mM, or about 15 mM to about 25 mM of a buffering agent. In exemplary aspects, the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM of a buffering agent.

Pharmaceutically acceptable buffering agents include without limitation, phosphate buffers, histidine, sodium citrate, HEPES, Tris, Bicine, glycine, N-glycylglycine, sodium acetate, sodium carbonate, glycyl glycine, lysine, arginine, sodium phosphate, and mixtures thereof. In some preferred embodiments, the buffer is a Tris buffer.

In exemplary aspects, the pharmaceutical composition of the present disclosure comprises about 5 mM to about 25 mM, about 5 mM to about 15 mM, about 10 mM to about 20 mM, or about 15 mM to about 25 mM Tris. In exemplary aspects, the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM Tris. In some embodiments, the pharmaceutical composition comprises about 8 mM Tris to about 22 mM Tris, or about 9 mM Tris to about 21 mM Tris, or about 10 mM Tris to about 20 mM Tris. In certain embodiments, the pharmaceutical composition comprises about 10 mM Tris.

In exemplary aspects, the pharmaceutical composition of the present disclosure comprises about 100 mM to about 250 mM, about 110 mM to about 240 mM, about 120 mM to about 230 mM, about 130 mM to about 220 mM, about 140 mM to about 210 mM, about 145 mM to about 205 mM, about 140 mM to about 200 mM, about 145 mM to about 200 mM, about 150 mM to about 200 mM, about 155 mM to about 195 mM, about 160 mM to about 195 mM, about 165 mM to about 195 mM, about 170 mM to about 195 mM, about 175 mM to about 195 mM, about 175 mM to about 194 mM, about 175 mM to about 193 mM, about 175 mM to about 192 mM, about 175 mM to about 191 mM, about 176 mM to about 190 mM, about 177 mM to about 189 mM, about 177 mM to about 188 mM, about 177 mM to about 187 mM, about 177 mM to about 186 mM, about 178 to about 184, about 178 to about 183, about 178 to about 182, about 179 to about 181 or about 180 mM of a pharmaceutically acceptable salt (as defined above). In exemplary embodiments, the composition comprises about 150 mM, about 155 mM, about 160 mM, about 175 mM, about 180 mM, about 185 mM, about 190 mM, or about 200 mM of a pharmaceutically acceptable salt. In certain embodiments, the pharmaceutically acceptable salt is a sodium salt (e.g., sodium chloride).

In certain embodiments, pharmaceutical compositions containing a non-ionic detergent are provided. Pharmaceutically acceptable non-ionic surfactants that may be used in the formulations disclosed herein are known in the art of pharmaceutical science, and include, without limitation, Polysorbate 80 (Tween 80; PS80), Polysorbate 20 (Tween 20; PS20), and various poloxamers or pluronics, including Pluronic F-68, and BRIJ 35, or mixtures thereof. In a preferred embodiment, the non-ionic surfactant used in the present pharmaceutical compositions is a pluronic, particularly Pluronic F-68.

In certain embodiments, the pharmaceutical composition of the present disclosure comprises about 0.001% (w/v) to about 0.01% (w/v) or about 0.0025% (w/v) to about 0.0075% (w/v) non-ionic surfactant. In exemplary aspects, the pharmaceutical composition comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), about 0.001% (w/v) non-ionic surfactant. In certain embodiments, the pharmaceutical composition of the present disclosure comprises about 0.005% (w/v) non-ionic surfactant.

In exemplary aspects, the pharmaceutical composition of the present disclosure comprises about 0.001% (w/v) to about 0.01% (w/v) or about 0.0025% (w/v) to about 0.0075% (w/v) Pluronic F68. In exemplary aspects, the pharmaceutical composition comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v), about 0.001% (w/v) Pluronic F68. In certain embodiments, the pharmaceutical composition of the present disclosure comprises about 0.005% (w/v) Pluronic F68.

In exemplary aspects, the pharmaceutical composition of the present disclosure comprises: adeno-associated virus (AAV) and about 5 mM to about 25 mM Tris, about 100 mM to about 250 mM sodium salt, about 0.001% (w/v) to about 0.01% (w/v) non-ionic surfactant, and a pH of between 7.0 and 9.0. In certain embodiments, the pharmaceutically acceptable salt is present at about 150 nM to about 210 mM. In certain embodiments, the pharmaceutically acceptable salt is present at about 170 nM to about 200 mM. In certain embodiments, the pharmaceutically acceptable salt is sodium chloride.

The present disclosure also provides a pharmaceutical composition that can be liquid or lyophilized (e.g., lyophilized from a liquid formulation) comprising adeno-associated virus (AAV) and about 10 mM Tris buffer, about 180 mM sodium chloride, about 0.005% (w/v) Pluronic F68 and the pH of the pharmaceutical composition is about 7.9±0.3.

pH

In exemplary embodiments, the pharmaceutical composition of the present disclosure has a pH of about 7.0, about 7.1, about 7.2, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In exemplary aspects, the pH of the pharmaceutical composition is above about 7.3 and below about 9.0 or below about 8.5. In certain embodiments, the pH of the pharmaceutical composition is about 7.8 to about 9.0 or is about 7.9. In other embodiments, the pH of the pharmaceutical composition is from about 7.3 to about 8.6.

### Additional Components

In some embodiments, the formulations or pharmaceutical compositions of the present disclosure comprise additional pharmaceutically acceptable ingredients. In exemplary aspects, the formulations or pharmaceutical compositions comprise any one or a combination of the following: acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. In some embodiments, the formulations or pharmaceutical compositions of the present disclosure comprise any one or a combination of the following components: acacia, acesulfame potassium, acetyltributyl citrate, acetyltriethyl citrate, agar, albumin, alcohol, dehydrated alcohol, denatured alcohol, dilute alcohol, aleuritic acid, alginic acid, aliphatic polyesters, alumina, aluminum hydroxide, aluminum stearate, amylopectin, α-amylose, ascorbic acid, ascorbyl palmitate, aspartame, bacteriostatic water for injection, bentonite, bentonite magma, benzalkonium chloride, benzethonium chloride, benzoic acid, benzyl alcohol, benzyl benzoate, bronopol, butylated hydroxyanisole, butylated hydroxytoluene, butylparaben, butylparaben sodium, calcium alginate, calcium ascorbate, calcium carbonate, calcium cyclamate, dibasic anhydrous calcium phosphate, dibasic dehydrate calcium phosphate, tribasic calcium phosphate, calcium propionate, calcium silicate, calcium sorbate, calcium stearate, calcium sulfate, calcium sulfate hemihydrate, canola oil, carbomer, carbon dioxide, carboxymethyl cellulose calcium, carboxymethyl cellulose sodium, β-carotene, carrageenan, castor oil, hydrogenated castor oil, cationic emulsifying wax, cellulose acetate, cellulose acetate phthalate, ethyl cellulose, microcrystalline cellulose, powdered cellulose, silicified microcrystalline cellulose, sodium carboxymethyl cellulose, cetostearyl alcohol, cetrimide, cetyl alcohol, chlorhexidine, chlorobutanol, chlorocresol, cholesterol, chlorhexidine acetate, chlorhexidine gluconate, chlorhexidine hydrochloride, chlorodifluoroethane (HCFC), chlorodifluoromethane, chlorofluorocarbons (CFC)chlorophenoxyethanol, chloroxylenol, corn syrup solids, anhydrous citric acid, citric acid monohydrate, cocoa butter, coloring agents, corn oil, cottonseed oil, cresol, m-cresol, o-cresol, p-cresol, croscarmellose sodium, crospovidone, cyclamic acid, cyclodextrins, dextrates, dextrin, dextrose, dextrose anhydrous, diazolidinyl urea, dibutyl phthalate, dibutyl sebacate, diethanolamine, diethyl phthalate, difluoroethane (HFC), dimethyl-β-cyclodextrin, cyclodextrin-type compounds such as Captisol®, dimethyl ether, dimethyl phthalate, dipotassium edentate, disodium edentate, disodium hydrogen phosphate, docusate calcium, docusate potassium, docusate sodium, dodecyl gallate, dodecyltrimethylammonium bromide, edentate calcium disodium, edtic acid, eglumine, ethyl alcohol, ethylcellulose, ethyl gallate, ethyl laurate, ethyl maltol, ethyl oleate, ethylparaben, ethylparaben potassium, ethylparaben sodium, ethyl vanillin, fructose, fructose liquid, fructose milled, fructose pyrogen-free, powdered fructose, fumaric acid, gelatin, glucose, liquid glucose, glyceride mixtures of saturated vegetable fatty acids, glycerin, glyceryl behenate, glyceryl monooleate, glyceryl monostearate, self-emulsifying glyceryl monostearate, glyceryl palmitostearate, glycine, glycols, glycofurol, guar gum, heptafluoropropane (HFC), hexadecyltrimethylammonium bromide, high fructose syrup, human serum albumin, hydrocarbons (HC), dilute hydrochloric acid, hydrogenated vegetable oil type II, hydroxyethyl cellulose, 2-hydroxyethyl-β-cyclodextrin, hydroxypropyl cellulose, low-substituted hydroxypropyl cellulose, 2-hydroxypropyl-β-cyclodextrin, hydroxypropyl methylcellulose, hydroxypropyl methylcellulose phthalate, imidurea, indigo carmine, ion exchangers, iron oxides, isopropyl alcohol, isopropyl myristate, isopropyl palmitate, isotonic saline, kaolin, lactic acid, lactitol, lactose, lanolin, lanolin alcohols, anhydrous lanolin, lecithin, magnesium aluminum silicate, magnesium carbonate, normal magnesium carbonate, magnesium carbonate anhydrous, magnesium carbonate hydroxide, magnesium hydroxide, magnesium lauryl sulfate, magnesium oxide, magnesium silicate, magnesium stearate, magnesium trisilicate, magnesium trisilicate anhydrous, malic acid, malt, maltitol, maltitol solution, maltodextrin, maltol, maltose, mannitol, medium chain triglycerides, meglumine, menthol, methylcellulose, methyl methacrylate, methyl oleate, methylparaben, methylparaben potassium, methylparaben sodium, microcrystalline cellulose and carboxymethylcellulose sodium, mineral oil, light mineral oil, mineral oil and lanolin alcohols, oil, olive oil, monoethanolamine, montmorillonite, octyl gallate, oleic acid, palmitic acid, paraffin, peanut oil, petrolatum, petrolatum and lanolin alcohols, pharmaceutical glaze, phenol, liquified phenol, phenoxyethanol, phenoxypropanol, phenylethyl alcohol, phenylmercuric acetate, phenylmercuric borate, phenylmercuric nitrate, polacrilin, polacrilin potassium, poloxamer, polydextrose, polyethylene glycol, polyethylene oxide, polyacrylates, polyethylene-polyoxypropylene-block polymers, polymethacrylates, polyoxyethylene alkyl ethers, polyoxyethylene castor oil derivatives, polyoxyethylene sorbitol fatty acid esters, polyoxyethylene stearates, polyvinyl alcohol, polyvinyl pyrrolidone, potassium alginate, potassium benzoate, potassium bicarbonate, potassium bisulfite, potassium chloride, potassium citrate, potassium citrate anhydrous, potassium hydrogen phosphate, potassium metabisulfite, monobasic potassium phosphate, potassium propionate, potassium sorbate, povidone, propanol, propionic acid, propylene carbonate, propylene glycol, propylene glycol alginate, propyl gallate, propylparaben, propylparaben potassium, propylparaben sodium, protamine sulfate, rapeseed oil, Ringer's solution, saccharin, saccharin ammonium, saccharin calcium, saccharin sodium, safflower oil, saponite, serum proteins, sesame oil, colloidal silica, colloidal silicon dioxide, sodium alginate, sodium ascorbate, sodium benzoate, sodium bicarbonate, sodium bisulfite, sodium chloride, anhydrous sodium citrate, sodium citrate dehydrate, sodium chloride, sodium cyclamate, sodium edentate, sodium dodecyl sulfate, sodium lauryl sulfate, sodium metabisulfite, sodium phosphate, dibasic, sodium phosphate, monobasic, sodium phosphate, tribasic, anhydrous sodium propionate, sodium propionate, sodium sorbate, sodium starch glycolate, sodium stearyl fumarate, sodium sulfite, sorbic acid, sorbitan esters (sorbitan fatty esters), sorbitol, sorbitol solution 70%, soybean oil, spermaceti wax, starch, corn starch, potato starch, pregelatinized starch, sterilizable maize starch, stearic acid, purified stearic acid, stearyl alcohol, sucrose, sugars, compressible sugar, confectioner's sugar, sugar spheres, invert sugar, Sugartab, Sunset Yellow FCF, synthetic paraffin, talc, tartaric acid, tartrazine, tetrafluoroethane (HFC), theobroma oil, thimerosal, titanium dioxide, alpha tocopherol, tocopheryl acetate, alpha tocopheryl acid succinate, beta-tocopherol, delta-tocopherol, gamma-tocopherol, tragacanth, triacetin, tributyl citrate, triethanolamine, triethyl citrate, trimethyl-β-cyclodextrin, trimethyltetradecylammonium bromide, tris buffer, trisodium edentate, vanillin, type I hydrogenated vegetable oil, water, soft water, hard water, carbon dioxide-free water, pyrogen-free water, water for injection, sterile water for inhalation, sterile water for injection, sterile water for irrigation, waxes, anionic emulsifying wax, carnauba wax, cationic emulsifying wax, cetyl ester wax, microcrystalline wax, nonionic emulsifying wax, suppository wax, white wax, yellow wax, white petrolatum, wool fat, xanthan gum, xylitol, zein, zinc propionate, zinc salts, zinc stearate, or any excipient in the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety for all intended purposes, discloses various components used in formulating pharmaceutically acceptable compositions and known techniques for the preparation thereof. Except insofar as any conventional agent is incompatible with the pharmaceutical compositions, its use in pharmaceutical compositions is contemplated.

In some aspects, the formulations or pharmaceutical compositions comprise a sugar or sugar alcohol. In some aspects, the sugar or sugar alcohol is sucrose, trehalose, mannitol, or a combination thereof.

In exemplary embodiments, the formulations or pharmaceutical compositions of the present disclosure do not comprise one or a combination of the above ingredients. In exemplary embodiments, the formulations or pharmaceutical compositions of the present disclosure comprises none of these ingredients. In exemplary aspects, the pharmaceutical composition of the present disclosure does not comprise dextran. In exemplary aspects, the pharmaceutical composition of the present disclosure does not comprise calcium chloride. In other exemplary aspects, the pharmaceutical composition of the present disclosure does not comprise a sugar or a sugar alcohol (e.g., does not comprise sucrose, trehalose, or mannitol). In related exemplary aspects, the pharmaceutical composition does not comprise glycine.

### AAV

In exemplary embodiments, the pharmaceutical composition of the present disclosure comprises AAV. The AAV may be of any AAV serotype. In exemplary aspects, the AAV is of AAV1 serotype, AAV2 serotype, AAV3 serotype, AAV4 serotype, AAV5 serotype, AAV6 serotype, AAV7 serotype, AAV8 serotype, AAV9 serotype, or AAV10 serotype. In exemplary aspects, the AAV is of AAV2 serotype or a variant thereof.

In certain embodiments, the AAV is an rAAV as described in U.S. Patent Publication No. 2020/0282077, which is incorporated herein by reference in its entirety, in particular an rAAV comprising a capsid protein according to paragraphs 171-179 of U.S. Patent Publication No. 2020/0282077 and/or comprising a heterologous nucleic acid according to paragraphs 222-248 of U.S. Patent Publication No. 2020/0282077.

In some aspects, the pharmaceutical composition comprises an rAAV comprising a variant AAV capsid encapsulating a heterologous nucleic acid encoding gene product, wherein the variant AAV capsid comprises a variant AAV capsid protein comprising an insertion of from about 7 to about 20 amino acids (a “heterologous peptide” or “peptide insertion”) in the GH-loop of a parental AAV capsid protein, wherein the peptide comprises the amino acid sequence ISDQTKH (SEQ ID NO:1). Preferably, the variant capsid protein, when present in an AAV virion, confers increased infectivity of a retinal cell compared to the infectivity of a retinal cell by an AAV virion comprising the corresponding parental capsid protein.

By the “GH loop,” or loop IV, of the AAV capsid protein it is meant the solvent-accessible portion referred to in the art as the GH loop, or loop IV, of AAV capsid protein. For the GH loop/loop IV of AAV capsid, see, e.g., van Vliet et al. (2006) Mol. Ther. 14:809; Padron et al. (2005) J. Virol. 79:5047; and Shen et al. (2007) Mol. Ther. 15:1955. Thus, for example, the insertion site can be within about amino acids 570-611 of AAV2 VP1.

In some embodiments, the peptide insertion has from 1 to 3 spacer amino acids (Y₁-Y₃) at the amino and/or carboxyl terminus of the amino acid sequence ISDQTKH (SEQ ID NO:1). Exemplary spacer amino acids include, without limitation, leucine (L), alanine (A), glycine (G), serine (S), threonine (T), and proline (P). In certain embodiments, a peptide insertion comprises 2 spacer amino acids at the N-terminus and 2 spacer amino acids at the C-terminus. In other embodiments, a peptide insertion comprises 2 spacer amino acids at the N-terminus and 1 spacer amino acids at the C-terminus. In preferred embodiments, the peptide insertion comprises or consists of the amino acid sequence LAISDQTKHA (SEQ ID NO:2).

In some aspects, the variant AAV capsid protein comprises a peptide insertion comprising the amino acid sequence ISDQTKH (SEQ ID NO:1) and further comprises one or more amino acid substitutions relative to a corresponding parental AAV capsid protein. Representative examples of amino acid substitutions may be found at e.g., col. 26, lines 40-65 of U.S. Pat. No. 11,576,983, the entire contents of which are incorporated herein by reference.

In some preferred embodiments, the variant AAV capsid protein comprises a peptide insertion comprising the amino acid sequence ISDQTKH (SEQ ID NO:1) and further comprises a P34A amino acid substitution relative to VP1 capsid of AAV2 or the corresponding substitution in another AAV serotype.

In other aspects, the variant capsid protein may comprise one or more features disclosed in U.S. Pat. No. 11,576,983, in particular, one or more features disclosed at column 26, line 66 to column 29, line 50 of U.S. Pat. No. 11,576,983.

In a particularly preferred embodiment, the variant capsid protein comprises the following amino acid sequence or comprises an amino acid sequence at least 80%, at least 90%, least 95%, at least 98%, or at least 99% identical to the following amino acid sequence:

(SEQ ID NO: 3)
MAADGYLPDWLEDTLSEGIRQWWKLKPGPPPPKAAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNLAISDQTKHARQA
ATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKEN
SKRWNPEIQYTSNYNKSVNVDFTVDINGVYSEPRPIGTRYLTRNL.

The variant AAV capsid protein of SEQ ID NO:3 contains the following modifications relative to native AAV2 capsid: (i) a proline (P) to alanine (A) mutation at amino acid position 34, which is located inside the assembled capsid (VP1 protein only), and (ii) an insertion of 10 amino acids (leucine-alanine-isoleucine-serine-aspartic acid-glutamine-threonine-lysine-histidine-alanine/LAISDQTKHA (SEQ ID NO:2)) at amino acid position 588, which is present in VP1, VP2, and VP3. In some embodiments, the capsid comprises a variant capsid protein comprising a sequence at least 90%, at least 95%, at least 98%, at least 99% identical to SEQ ID NO:3 and comprising a P34A substitution and an LAISDQTKHA (SEQ ID NO:2) peptide insertion at amino acid position 588.

In some aspects, a pharmaceutical composition as herein described comprises an rAAV encapsulating a heterologous nucleic acid comprising a nucleotide sequence encoding one or more gene products. In some aspects, the one or more gene products are selected from an interfering RNA (e.g., a microRNA) and a polypeptide. In particularly preferred embodiments, the heterologous nucleic acid encodes aflibercept and optionally further encodes an interfering RNA that decreases the expression of VEGF-C. In related aspects, the heterologous nucleic acid comprises a nucleotide sequence described in U.S. Patent Publication No. 2024/0131195A1, the entire contents of which are incorporated herein by reference, in particular at Table 2.

In some aspects, the rAAV comprises a heterologous nucleic acid comprising the following nucleotide sequence encoding aflibercept, codon-optimized for expression in humans:

(SEQ ID NO: 4)
ATGGTTTCTTACTGGGACACCGGCGTGCTGCTGTGTGCCCTGCTTTCTTG
TCTGCTGCTGACCGGCTCTAGCAGCGGCTCTGATACCGGCAGACCCTTCG
TGGAAATGTACAGCGAGATCCCCGAGATCATCCACATGACCGAGGGCAGA
GAGCTGGTCATCCCTTGCAGAGTGACAAGCCCCAACATCACCGTGACTCT
GAAGAAGTTCCCTCTGGACACACTGATCCCCGACGGCAAGAGAATCATCT
GGGACAGCCGGAAGGGCTTCATCATCAGCAACGCCACCTACAAAGAGATC
GGCCTGCTGACCTGTGAAGCCACCGTGAATGGCCACCTGTACAAGACCAA
CTACCTGACACACAGACAGACCAACACCATCATCGACGTGGTGCTGAGCC
CTAGCCACGGCATTGAACTGTCTGTGGGCGAGAAGCTGGTGCTGAACTGT
ACCGCCAGAACCGAGCTGAACGTGGGCATCGACTTCAACTGGGAGTACCC
CAGCAGCAAGCACCAGCACAAGAAACTGGTCAACCGGGACCTGAAAACCC
AGAGCGGCAGCGAGATGAAGAAATTCCTGAGCACCCTGACCATCGACGGC
GTGACCAGAAGTGACCAGGGCCTGTACACATGTGCCGCCAGCTCTGGCCT
GATGACCAAGAAAAACAGCACCTTCGTGCGGGTGCACGAGAAGGACAAGA
CCCACACCTGTCCTCCATGTCCTGCTCCAGAACTGCTCGGCGGACCTTCC
GTGTTCCTGTTTCCTCCAAAGCCTAAGGACACCCTGATGATCAGCAGAAC
CCCTGAAGTGACCTGCGTGGTGGTGGATGTGTCCCACGAGGATCCCGAAG
TGAAGTTCAATTGGTACGTGGACGGCGTGGAAGTGCACAACGCCAAGACC
AAGCCTAGAGAGGAACAGTACAATAGCACCTACAGAGTGGTGTCCGTGCT
GACCGTGCTGCACCAGGATTGGCTGAACGGCAAAGAGTACAAGTGCAAGG
TGTCCAACAAGGCCCTGCCTGCTCCTATCGAGAAAACCATCTCCAAGGCC
AAGGGCCAGCCTAGGGAACCCCAGGTTTACACACTGCCTCCAAGCAGGGA
CGAGCTGACAAAGAACCAGGTGTCCCTGACCTGCCTGGTCAAGGGCTTCT
ACCCTTCCGATATCGCCGTGGAATGGGAGAGCAATGGCCAGCCTGAGAAC
AACTACAAGACAACCCCTCCTGTGCTGGACAGCGACGGCTCATTCTTCCT
GTACAGCAAGCTGACAGTGGACAAGAGCAGATGGCAGCAGGGCAACGTGT
TCAGCTGCTCCGTGATGCACGAGGCCCTGCACAACCACTACACCCAGAAG
TCCCTGAGCCTGTCTCCTGGCAAA

In some embodiments, the sequence is at least 80%, at least 90%, at least 95% or at least 99% identical to the nucleotide sequence of SEQ ID NO:4 and/or comprises a stop codon (e.g. TGA) at the end of the sequence. In some embodiments, the aflibercept gene product comprises the following amino acid sequence or a sequence at least 90%, 95%, 97%, 98%, or at least 99% identical thereto:

(SEQ ID NO: 5)
MVSYWDTGVLLCALLSCLLLTGSSSGSDTGRPFVEMYSEIPEIIHMTEGR
ELVIPCRVTSPNITVTLKKFPLDTLIPDGKRIIWDSRKGFIISNATYKEI
GLLTCEATVNGHLYKTNYLTHRQTNTIIDVVLSPSHGIELSVGEKLVLNC
TARTELNVGIDFNWEYPSSKHQHKKLVNRDLKTQSGSEMKKFLSTLTIDG
VTRSDQGLYTCAASSGLMTKKNSTFVRVHEKDKTHTCPPCPAPELLGGPS
VFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQK
SLSLSPGK

In related aspects, the pharmaceutical composition comprises an rAAV comprising a heterologous nucleic acid comprising the following sequence (encoding aflibercept+human VEGF-C interfering RNA) or a sequence at least 80%, at least 90%, at least 95%, at least 98% or at least 99% identical thereto:

(SEQ ID NO: 6)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTATCGA
TTGAATTCCCCGGGGATCCACTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTCGAGGTGAGCCCC
ACGTTCTGCTTCACTCTCCCCATCTCCCCCCCCTCCCCACCCCCAATTTT
GTATTTATTTATTTTTTAATTATTTTGTGCAGCGATGGGGGCGGGGGGGG
GGGGGGGGCGCGCGCCAGGCGGGGCGGGGGGGGGCGAGGGGCGGGGCGGG
GCGAGGCGGAGAGGTGCGGCGGCAGCCAATCAGAGCGGCGCGCTCCGAAA
GTTTCCTTTTATGGCGAGGCGGCGGCGGCGGCGGCCCTATAAAAAGCGAA
GCGCGCGGCGGGCGGGGAGTCGCTGCGACGCTGCCTTCGCCCCGTGCCCC
GCTCCGCCGCCGCCTCGCGCCGCCCGCCCCGGCTCTGACTGACCGCGTTA
CTCCCACAGGTGAGCGGGCGGGACGGCCCTTCTCCTCCGGGCTGTAATTA
GCGCTTGGTTTAATGACGGCTTGTTTCTTTTCTGTGGCTGCGTGAAAGCC
TTGAGGGGCTCCGGGAGGGCCCTTTGTGCGGGGGGAGCGGCTCGGGGGGT
GCGTGCGTGTGTGTGTGCGTGGGGAGCGCCGCGTGCGGCTCCGCGCTGCC
CGGCGGCTGTGAGCGCTGCGGGCGCGGCGCGGGGCTTTGTGCGCTCCGCA
GTGTGCGCGAGGGGAGCGCGGCCGGGGGCGGTGCCCCGCGGTGCGGGGGG
GGCTGCGAGGGGAACAAAGGCTGCGTGCGGGGTGTGTGCGTGGGGGGGTG
AGCAGGGGGTGTGGGCGCGTCGGTCGGGCTGCAACCCCCCCTGCACCCCC
CTCCCCGAGTTGCTGAGCACGGCCCGGCTTCGGGTGCGGGGCTCCGTACG
GGGCGTGGCGCGGGGCTCGCCGTGCCGGGCGGGGGGTGGCGGCAGGTGGG
GGTGCCGGGCGGGGCGGGGCCGCCTCGGGCCGGGGAGGGCTCGGGGGAGG
GGCGCGGCGGCCCCCGGAGCGCCGGCGCGACTTCTTAACCCAACAGAAGG
CTCGAGAAGGTATATTGCTGTTGACAGTGAGCGGCTACCTCAGCAAGACG
TTATTTAGTGAAGCCACAGATGTAAATAACGTCTTGCTGAGGTAGCTGCC
TACTGCCTCGGACTTCAAGGGGCTAGAATTCGCGGCTGTCGAGGCGCGGC
GAGCCGCAGCCATTGCCTTTTATGGTAATCGTGCGAGAGGGCGCAGGGAC
TTCCTTTGTCCCAAATCTGTGCGGAGCCGAAATCTGGGAGGCGCCGCCGC
ACCCCCTCTAGCGGGCGCGGGGCGAAGCGGTGCGGCGCCGGCAGGAAGGA
AATGGGCGGGGAGGGCCTTCGTGCGTCGCCGCGCCGCCGTCCCCTTCTCC
CTCTCCAGCCTCGGGGCTGTCCGCGGGGGGACGGCTGCCTTCGGGGGGGA
CGGGGCAGGGCGGGGTTCGGCTTCTGGCGTGTGACCGGCGGCTCTAGAGC
CTCTGCTAACCATGTTCATGCCTTCTTCTTTTTCCTACAGTCTAGAGTCG
ACCTGCAGGTGGATATCTTGGCTAGCACGCCACCATGGTTTCTTACTGGG
ACACCGGCGTGCTGCTGTGTGCCCTGCTTTCTTGTCTGCTGCTGACCGGC
TCTAGCAGCGGCTCTGATACCGGCAGACCCTTCGTGGAAATGTACAGCGA
GATCCCCGAGATCATCCACATGACCGAGGGCAGAGAGCTGGTCATCCCTT
GCAGAGTGACAAGCCCCAACATCACCGTGACTCTGAAGAAGTTCCCTCTG
GACACACTGATCCCCGACGGCAAGAGAATCATCTGGGACAGCCGGAAGGG
CTTCATCATCAGCAACGCCACCTACAAAGAGATCGGCCTGCTGACCTGTG
AAGCCACCGTGAATGGCCACCTGTACAAGACCAACTACCTGACACACAGA
CAGACCAACACCATCATCGACGTGGTGCTGAGCCCTAGCCACGGCATTGA
ACTGTCTGTGGGCGAGAAGCTGGTGCTGAACTGTACCGCCAGAACCGAGC
TGAACGTGGGCATCGACTTCAACTGGGAGTACCCCAGCAGCAAGCACCAG
CACAAGAAACTGGTCAACCGGGACCTGAAAACCCAGAGCGGCAGCGAGAT
GAAGAAATTCCTGAGCACCCTGACCATCGACGGCGTGACCAGAAGTGACC
AGGGCCTGTACACATGTGCCGCCAGCTCTGGCCTGATGACCAAGAAAAAC
AGCACCTTCGTGCGGGTGCACGAGAAGGACAAGACCCACACCTGTCCTCC
ATGTCCTGCTCCAGAACTGCTCGGCGGACCTTCCGTGTTCCTGTTTCCTC
CAAAGCCTAAGGACACCCTGATGATCAGCAGAACCCCTGAAGTGACCTGC
GTGGTGGTGGATGTGTCCCACGAGGATCCCGAAGTGAAGTTCAATTGGTA
CGTGGACGGCGTGGAAGTGCACAACGCCAAGACCAAGCCTAGAGAGGAAC
AGTACAATAGCACCTACAGAGTGGTGTCCGTGCTGACCGTGCTGCACCAG
GATTGGCTGAACGGCAAAGAGTACAAGTGCAAGGTGTCCAACAAGGCCCT
GCCTGCTCCTATCGAGAAAACCATCTCCAAGGCCAAGGGCCAGCCTAGGG
AACCCCAGGTTTACACACTGCCTCCAAGCAGGGACGAGCTGACAAAGAAC
CAGGTGTCCCTGACCTGCCTGGTCAAGGGCTTCTACCCTTCCGATATCGC
CGTGGAATGGGAGAGCAATGGCCAGCCTGAGAACAACTACAAGACAACCC
CTCCTGTGCTGGACAGCGACGGCTCATTCTTCCTGTACAGCAAGCTGACA
GTGGACAAGAGCAGATGGCAGCAGGGCAACGTGTTCAGCTGCTCCGTGAT
GCACGAGGCCCTGCACAACCACTACACCCAGAAGTCCCTGAGCCTGTCTC
CTGGCAAATGAGCCACGCGTAACACGTGGGGGATCCAGACATGATAAGAT
ACATTGATGAGTTTGGACAAACCACAACTAGAATGCAGTGAAAAAAATGC
TTTATTTGTGAAATTTGTGATGCTATTGCTTTATTTGTAACCATTATAAG
CTGCAATAAACAAGTTAACAACAACAATTGCATTCATTTTATGTTTCAGG
TTCAGGGGGAGGTGTGGGAGGTTTTTTAAAGCAAGTAAAACCTCTACAAA
TGTGGTATGGCTGATTATGATCAATGCATGGCCGGCCGGAGGAACCCCTA
GTGATGGAGTTGGCCACTCCCTCTCTGCGCGCTCGCTCGCTCACTGAGGC
CGCCCGGGCAAAGCCCGGGCGTCGGGCGACCTTTGGTCGCCCGGCCTCAG
TGAGCGAGCGAGCGCGCAGAGAGGGAGTGGCCAA.

In related aspects, the pharmaceutical composition comprises an rAAV comprising a heterologous nucleic acid comprising a nucleotide sequence encoding a wild type or codon-optimized human Rab escort protein-1 (REP1) protein, e.g., as described in U.S. Pat. No. 11,357,870, the entire contents of which are hereby incorporated by reference. In some preferred aspects, the heterologous nucleic acid encoding REP1 comprises the following codon-optimized nucleotide sequence or a sequence at least 90%, at least 91%, at least 92%, at least 93%, at least 94%, at least 95%, at least 97%, at least 98% or at least 99% identical thereto:

(SEQ ID NO: 7)
ATGGCTGATACACTGCCTTCTGAGTTTGATGTGATCGTGATTGGAACTGG
ACTGCCTGAGAGTATTATTGCTGCTGCTTGTAGTAGAAGCGGCCGGAGAG
TGCTGCACGTGGACAGCAGATCCTACTATGGCGGCAACTGGGCCTCTTTC
AGCTTTTCCGGCCTGCTGAGCTGGCTGAAGGAGTACCAGGAGAACTCCGA
CATCGTGTCTGATAGCCCCGTGTGGCAGGACCAGATCCTGGAGAATGAGG
AGGCCATCGCCCTGTCCAGGAAGGATAAGACCATCCAGCACGTGGAGGTG
TTCTGCTATGCCAGCCAGGACCTGCACGAGGATGTGGAGGAGGCAGGCGC
CCTGCAGAAGAACCACGCCCTGGTGACCTCCGCCAATTCTACAGAGGCCG
CCGACTCCGCCTTTCTGCCTACCGAGGATGAGTCCCTGTCTACAATGTCT
TGTGAGATGCTGACCGAGCAGACACCTAGCTCCGATCCAGAGAACGCCCT
GGAGGTCAATGGCGCCGAGGTGACCGGCGAGAAGGAGAACCACTGCGACG
ATAAGACCTGCGTGCCAAGCACATCCGCCGAGGACATGTCCGAGAACGTG
CCTATCGCCGAGGATACCACAGAGCAGCCAAAGAAGAATCGCATCACATA
CAGCCAGATCATCAAGGAGGGCAGGCGCTTCAATATCGACCTGGTGTCTA
AGCTGCTGTACAGCCGGGGCCTGCTGATCGATCTGCTGATCAAGAGCAAC
GTGTCCCGCTATGCCGAGTTCAAGAATATCACCAGAATCCTGGCCTTTCG
GGAGGGAAGAGTGGAGCAGGTGCCCTGCAGCAGAGCCGACGTGTTCAACT
CCAAGCAGCTGACAATGGTGGAGAAGAGGATGCTGATGAAGTTCCTGACA
TTTTGTATGGAGTACGAGAAGTATCCAGATGAGTACAAGGGCTATGAGGA
GATCACCTTTTACGAGTATCTGAAGACCCAGAAGCTGACACCCAATCTGC
AGTACATCGTGATGCACTCCATCGCCATGACCTCTGAGACAGCCTCTAGC
ACCATCGACGGCCTGAAGGCCACAAAGAACTTCCTGCACTGCCTGGGCCG
GTACGGCAATACACCCTTCCTGTTTCCTCTGTATGGCCAGGGCGAGCTGC
CCCAGTGCTTCTGTAGAATGTGCGCCGTGTTTGGCGGCATCTATTGCCTG
AGGCACTCTGTGCAGTGTCTGGTGGTGGACAAGGAGAGCCGCAAGTGTAA
GGCCATCATCGATCAGTTTGGCCAGCGGATCATCTCTGAGCACTTCCTGG
TGGAGGACAGCTACTTTCCTGAGAACATGTGCTCCAGGGTGCAGTATCGC
CAGATCAGCCGGGCCGTGCTGATCACCGATAGATCCGTGCTGAAGACAGA
CAGCGATCAGCAGATCAGCATCCTGACCGTGCCAGCAGAGGAGCCAGGCA
CCTTCGCCGTGAGAGTGATCGAGCTGTGCTCCTCTACCATGACATGTATG
AAGGGCACCTACCTGGTGCACCTGACCTGCACAAGCTCCAAGACAGCCCG
CGAGGACCTGGAGAGCGTGGTGCAGAAGCTGTTCGTGCCCTACACCGAGA
TGGAGATCGAGAACGAGCAGGTGGAGAAGCCTAGAATCCTGTGGGCCCTG
TACTTCAACATGAGAGACTCTAGCGATATCTCTAGGAGCTGTTACAACGA
TCTGCCCTCTAACGTGTACGTGTGCAGCGGACCTGACTGTGGCCTGGGAA
ACGATAATGCCGTGAAGCAGGCCGAGACACTGTTCCAGGAGATTTGCCCT
AACGAGGACTTTTGTCCCCCTCCACCCAATCCAGAGGATATCATCCTGGA
CGGCGATTCCCTGCAGCCAGAGGCCTCTGAGTCCTCTGCCATCCCCGAGG
CCAATAGCGAAACATTCAAAGAAAGCACAAATCTGGGAAACCTGGAAGAA
AGTAGTGAGTAA

In other related aspects, the pharmaceutical composition comprises an rAAV comprising a heterologous nucleic acid comprising a nucleotide sequence encoding a wild type or codon-optimized human Retinitis Pigmentosa GTPase Regulator (RPGR) protein, e.g., as described in U.S. Pat. No. 11,345,930, the entire contents of which are hereby incorporated by reference. In some preferred aspects, the heterologous nucleic acid encodes human RPGR ORF15 and comprises the following codon-optimized nucleotide sequence or a sequence at least 90%, at least 91%, at least 92%, at least 93%, at least 94%, at least 95%, at least 97%, at least 98% or at least 99% identical thereto:

(SEQ ID NO: 8)
ATGAGAGAACCCGAGGAACTGATGCCCGACTCTGGCGCCGTGTTTACCTT
CGGCAAGAGCAAGTTCGCCGAGAACAACCCCGGCAAGTTCTGGTTCAAGA
ACGACGTGCCAGTGCACCTGAGCTGCGGAGATGAACACTCTGCCGTGGTC
ACCGGCAACAACAAGCTGTACATGTTCGGCAGCAACAACTGGGGCCAGCT
CGGCCTGGGATCTAAGTCTGCCATCAGCAAGCCTACCTGCGTGAAGGCCC
TGAAGCCTGAGAAAGTGAAACTGGCCGCCTGCGGCAGAAATCACACCCTG
GTTTCTACCGAAGGCGGCAATGTGTATGCCACCGGCGGAAACAATGAGGG
ACAGCTTGGACTGGGCGACACCGAGGAAAGAAACACCTTCCACGTGATCA
GCTTTTTCACCAGCGAGCACAAGATCAAGCAGCTGAGCGCCGGCTCTAAT
ACCTCTGCCGCTCTGACAGAGGACGGCAGACTGTTTATGTGGGGCGACAA
TTCTGAGGGCCAGATCGGACTGAAGAACGTGTCCAATGTGTGCGTGCCCC
AGCAAGTGACAATCGGCAAGCCTGTGTCTTGGATCAGCTGCGGCTACTAC
CACAGCGCCTTTGTGACAACCGATGGCGAGCTGTATGTGTTCGGCGAGCC
AGAGAATGGCAAGCTGGGACTGCCTAACCAGCTGCTGGGCAATCACAGAA
CCCCTCAGCTGGTGTCTGAGATCCCCGAAAAAGTGATCCAGGTGGCCTGT
GGCGGAGAGCACACAGTGGTGCTGACAGAGAATGCCGTGTACACCTTTGG
CCTGGGCCAGTTTGGACAACTCGGACTGGGAACCTTCCTGTTCGAGACAA
GCGAGCCCAAAGTGATCGAGAACATCCGGGACCAGACCATCAGCTACATC
AGCTGTGGCGAGAACCACACAGCCCTGATCACAGACATCGGCCTGATGTA
CACATTCGGCGACGGAAGGCATGGAAAGCTCGGACTTGGCCTGGAAAACT
TCACCAACCACTTCATCCCTACGCTGTGCAGCAACTTCCTGCGGTTCATT
GTGAAGCTGGTGGCCTGCGGAGGATGCCACATGGTGGTTTTTGCTGCCCC
TCACAGAGGCGTGGCCAAAGAGATTGAGTTCGACGAGATCAACGATACCT
GCCTGAGCGTGGCCACCTTCCTGCCTTACAGCAGCCTGACATCTGGCAAC
GTGCTGCAGAGGACACTGAGCGCCAGAATGCGCAGACGGGAAAGAGAGAG
AAGCCCCGACAGCTTCAGCATGAGAAGAACCCTGCCTCCAATCGAGGGCA
CACTGGGCCTGTCTGCCTGCTTTCTGCCTAACAGCGTGTTCCCCAGATGC
AGCGAGAGAAACCTGCAAGAGAGCGTGCTGAGCGAGCAGGATCTGATGCA
GCCTGAGGAACCCGACTACCTGCTGGACGAGATGACCAAAGAGGCCGAGA
TCGACAACAGCAGCACAGTGGAAAGCCTGGGCGAGACAACCGACATCCTG
AACATGACCCACATCATGAGCCTGAACAGCAACGAGAAGTCTCTGAAGCT
GAGCCCCGTGCAGAAGCAGAAGAAGCAGCAGACCATCGGCGAGCTGACAC
AGGATACTGCCCTGACCGAGAACGACGACAGCGACGAGTACGAAGAGATG
AGCGAGATGAAGGAAGGCAAGGCCTGCAAGCAGCACGTGTCCCAGGGCAT
CTTTATGACCCAGCCTGCCACCACCATCGAGGCCTTTTCCGACGAGGAAG
TGGAAATCCCCGAGGAAAAAGAGGGCGCCGAGGACAGCAAAGGCAACGGC
ATTGAGGAACAAGAGGTGGAAGCCAACGAAGAGAACGTGAAGGTGCACGG
CGGACGGAAAGAAAAGACCGAGATCCTGAGCGACGACCTGACCGATAAGG
CCGAGGTTTCCGAGGGCAAAGCCAAGTCTGTGGGAGAAGCCGAGGATGGA
CCTGAAGGCCGCGGAGATGGAACCTGTGAAGAAGGATCTAGCGGAGCCGA
GCACTGGCAGGATGAGGAACGCGAGAAGGGCGAGAAAGACAAAGGCAGAG
GCGAGATGGAAAGACCCGGCGAGGGCGAAAAAGAGCTGGCCGAGAAAGAG
GAATGGAAGAAACGCGACGGCGAAGAACAAGAGCAGAAAGAAAGAGAGCA
GGGCCACCAGAAAGAACGGAATCAAGAGATGGAAGAAGGCGGCGAGGAAG
AACACGGCGAAGGGGAAGAAGAGGAAGGCGACCGAGAGGAAGAAGAAGAG
AAAGAAGGCGAAGGCAAAGAAGAAGGCGAGGGCGAAGAGGTGGAAGGCGA
GCGTGAAAAAGAAGAGGGCGAACGCAAGAAAGAAGAACGCGCCGGAAAAG
AGGAAAAAGGCGAGGAAGAGGGCGACCAAGGCGAAGGCGAGGAAGAAGAA
ACTGAAGGCAGAGGGGAAGAGAAAGAGGAAGGCGGCGAAGTCGAAGGCGG
AGAGGTTGAAGAAGGCAAAGGCGAGCGAGAAGAGGAAGAAGAAGAAGGCG
AAGGCGAGGAAGAGGAAGGCGAAGGCGAAGAGGAAGAAGGCGAAGGGGAA
GAAGAAGAAGGCGAAGGCAAGGGCGAAGAGGAGGGCGAAGAAGGCGAGGG
CGAAGAGGAGGGCGAAGAAGGCGAAGGCGAGGGCGAAGAAGAAGAAGGCG
AAGGCGAAGGCGAGGAAGAAGGCGAAGGCGAAGGGGAAGAAGAGGAAGGC
GAAGGCGAAGGCGAAGAAGAAGGCGAAGGCGAGGGCGAAGAGGAAGAAGG
CGAAGGCAAAGGGGAAGAAGAAGGCGAGGAAGGCGAAGGCGAAGGCGAGG
AAGAAGAAGGCGAAGGCGAGGGCGAAGATGGCGAAGGCGAAGGCGAAGAG
GAAGAGGGCGAGTGGGAGGGCGAAGAAGAGGAAGGCGAAGGCGAGGGCGA
AGAGGAAGGCGAAGGCGAGGGCGAAGAAGGCGAAGGCGAAGGCGAGGAAG
AGGAAGGCGAAGGCGAAGGGGAAGAAGAAGAGGGCGAAGAAGAAGGCGAA
GAGGAAGGCGAAGGGGAAGAAGAAGGCGAAGGCGAAGGCGAAGAAGAGGA
AGAGGGCGAAGTTGAAGGCGAGGTTGAGGGCGAAGAAGGCGAAGGCGAAG
GGGAAGAAGAAGAAGGCGAGGAAGAAGGGGAAGAGAGAGAAAAAGAAGGC
GAGGGCGAAGAAAACCGCCGGAACCGCGAAGAGGAAGAGGAAGAAGAGGG
CAAGTACCAAGAGACTGGCGAGGAAGAGAACGAGCGGCAGGATGGCGAAG
AGTACAAGAAGGTGTCCAAGATCAAGGGCAGCGTGAAGTACGGCAAGCAC
AAGACCTACCAGAAGAAGTCCGTCACCAACACGCAAGGCAATGGAAAAGA
ACAGCGGAGCAAGATGCCCGTGCAGTCCAAGAGGCTGCTGAAGAATGGCC
CTAGCGGCAGCAAGAAATTCTGGAACAATGTGCTGCCCCACTACCTCGAG
CTGAAGTGA

In other related aspects, the pharmaceutical composition comprises an rAAV comprising a heterologous nucleic acid comprising a nucleotide sequence encoding an engineered human complement regulator factor H (fH) gene operably linked to an expression control sequence, wherein the human fH (hfH) gene encodes a soluble hfH protein variant that retains complement regulatory function, wherein said fH variant comprises short consensus repeats (SCRs) 1, 2, 3, 4, 19 and 20. In some aspects, the soluble hfH protein variant comprises an amino acid sequence as set forth in U.S. Pat. No. 10,988,519, the entire contents of which are incorporated herein by reference. In a particularly preferred embodiment, the heterologous nucleic acid encodes an fH variant having the following amino acid sequence:

(SEQ ID NO: 9)
MRLLAKIICLMLWAICVAEDCNELPPRRNTEILTGSWSDQTYPEGTQAIY
KCRPGYRSLGNIIMVCRKGEWVALNPLRKCQKRPCGHPGDTPFGTFTLTG
GNVFEYGVKAVYTCNEGYQLLGEINYRECDTDGWTNDIPICEVVKCLPVT
APENGKIVSSAMEPDREYHFGQAVRFVCNSGYKIEGDEEMHCSDDGFWSK
EKPKCVEISCKSPDVINGSPISQKIIYKENERFQYKCNMGYEYSERGDAV
CTESGWRPLPSCEEKSTLKPCDYPDIKHGGLYHENMRRPYFPVAVGKYYS
YYCDEHFETPSGSYWDHIHCTQDGWSPAVPCLRKCYFPYLENGYNQNYGR
KFVQGKSIDVACHPGYALPKAQTTVTCMENGWSPTPRCIRVKTCSKSSID
IENGFISESQYTYALKEKAKYQCKLGYVTADGETSGSITCGKDGWSAQPT
CIKSIKTDCLSLPSFENAIPMGEKKDVYKAGEQVTYTCATYYKMDGASNV
TCINSRWTGRPTCRDTSCVNPPTVQNAYIVSRQMSKYPSGERVRYQCRSP
YEMFGDEEVMCLNGNWTEPPQCKDSTGKCGPPPPIDNGDITSFPLSVYAP
ASSVEYQCQNLYQLEGNKRITCRNGQWSEPPKCLHPCVISREIMENYNIA
LRWTAKQKLYSRTGESVEFVCKRGYRLSSRSHTLRTTCWDGKLEYPTCAK
R

In some aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 10)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTATCGA
TTGAATTCCCCGGGGATCCACTAGTTATTAATAGTAATCAATTACGGGGT
CATTAGTTCATAGCCCATATATGGAGTTCCGCGTTACATAACTTACGGTA
AATGGCCCGCCTGGCTGACCGCCCAACGACCCCCGCCCATTGACGTCAAT
AATGACGTATGTTCCCATAGTAACGCCAATAGGGACTTTCCATTGACGTC
AATGGGTGGAGTATTTACGGTAAACTGCCCACTTGGCAGTACATCAAGTG
TATCATATGCCAAGTACGCCCCCTATTGACGTCAATGACGGTAAATGGCC
CGCCTGGCATTATGCCCAGTACATGACCTTATGGGACTTTCCTACTTGGC
AGTACATCTACGTATTAGTCATCGCTATTACCATGGTCGAGGTGAGCCCC
ACGTTCTGCTTCACTCTCCCCATCTCCCCCCCCTCCCCACCCCCAATTTT
GTATTTATTTATTTTTTAATTATTTTGTGCAGCGATGGGGGCGGGGGGGG
GGGGGGGGCGCGCGCCAGGCGGGGGGGGGGGGGCGAGGGGCGGGGCGGGG
CGAGGCGGAGAGGTGCGGCGGCAGCCAATCAGAGCGGCGCGCTCCGAAAG
TTTCCTTTTATGGCGAGGCGGCGGCGGCGGCGGCCCTATAAAAAGCGAAG
CGCGCGGCGGGCGGGGAGTCGCTGCGACGCTGCCTTCGCCCCGTGCCCCG
CTCCGCCGCCGCCTCGCGCCGCCCGCCCCGGCTCTGACTGACCGCGTTAC
TCCCACAGGTGAGCGGGCGGGACGGCCCTTCTCCTCCGGGCTGTAATTAG
CGCTTGGTTTAATGACGGCTTGTTTCTTTTCTGTGGCTGCGTGAAAGCCT
TGAGGGGCTCCGGGAGGGCCCTTTGTGCGGGGGGAGCGGCTCGGGGGGTG
CGTGCGTGTGTGTGTGCGTGGGGAGCGCCGCGTGCGGCTCCGCGCTGCCC
GGCGGCTGTGAGCGCTGCGGGCGCGGCGCGGGGCTTTGTGCGCTCCGCAG
TGTGCGCGAGGGGAGCGCGGCCGGGGGCGGTGCCCCGCGGTGCGGGGGGG
GCTGCGAGGGGAACAAAGGCTGCGTGCGGGGTGTGTGCGTGGGGGGGTGA
GCAGGGGGTGTGGGCGCGTCGGTCGGGCTGCAACCCCCCCTGCACCCCCC
TCCCCGAGTTGCTGAGCACGGCCCGGCTTCGGGTGCGGGGCTCCGTACGG
GGCGTGGCGCGGGGCTCGCCGTGCCGGGCGGGGGGTGGCGGCAGGTGGGG
GTGCCGGGCGGGGCGGGGCCGCCTCGGGCCGGGGAGGGCTCGGGGGAGGG
GCGCGGCGGCCCCCGGAGCGCCGGCGGCTGTCGAGGCGCGGCGAGCCGCA
GCCATTGCCTTTTATGGTAATCGTGCGAGAGGGCGCAGGGACTTCCTTTG
TCCCAAATCTGTGCGGAGCCGAAATCTGGGAGGCGCCGCCGCACCCCCTC
TAGCGGGCGCGGGGCGAAGCGGTGCGGCGCCGGCAGGAAGGAAATGGGCG
GGGAGGGCCTTCGTGCGTCGCCGCGCCGCCGTCCCCTTCTCCCTCTCCAG
CCTCGGGGCTGTCCGCGGGGGGACGGCTGCCTTCGGGGGGGACGGGGCAG
GGCGGGGTTCGGCTTCTGGCGTGTGACCGGCGGCTCTAGAGCCTCTGCTA
ACCATGTTCATGCCTTCTTCTTTTTCCTACAGTCTAGAGTCGACCTGCAG
GTGGATATCTTGCTAGCACGCCACCATGCGCCTGCTGGCCAAGATCATCT
GCCTGATGCTGTGGGCCATCTGCGTGGCCGAGGACTGTAACGAGCTGCCC
CCTCGGAGAAATACAGAGATCCTGACCGGCTCTTGGAGCGATCAGACCTA
CCCAGAGGGCACACAGGCCATCTACAAGTGCAGGCCCGGCTATCGCTCCC
TGGGCAATATCATCATGGTGTGCAGGAAGGGAGAGTGGGTGGCCCTGAAC
CCACTGAGAAAGTGCCAGAAGAGGCCATGTGGACACCCAGGCGACACACC
TTTCGGCACCTTTACACTGACCGGCGGCAACGTGTTCGAGTACGGCGTGA
AGGCCGTGTATACCTGCAATGAGGGCTACCAGCTGCTGGGCGAGATCAAC
TACCGGGAGTGTGACACAGATGGCTGGACCAATGATATCCCCATCTGCGA
GGTGGTGAAGTGTCTGCCAGTGACCGCCCCCGAGAACGGCAAGATCGTGA
GCTCCGCCATGGAGCCTGACAGAGAGTATCACTTCGGCCAGGCCGTGAGG
TTCGTGTGCAATAGCGGCTACAAGATCGAGGGCGATGAGGAGATGCACTG
TTCCGACGATGGCTTCTGGTCTAAGGAGAAGCCCAAGTGCGTGGAGATCT
CCTGTAAGTCTCCTGACGTGATTAACGGCAGCCCAATCTCCCAGAAGATC
ATCTATAAGGAGAATGAGCGGTTTCAGTACAAGTGCAACATGGGCTACGA
GTATTCCGAGAGAGGCGATGCCGTGTGCACAGAGTCTGGATGGAGGCCCC
TGCCTAGCTGCGAGGAGAAGTCCACCCTGAAGCCTTGTGACTATCCAGAT
ATCAAGCACGGCGGCCTGTATCACGAGAATATGAGGCGCCCTTACTTCCC
AGTGGCCGTGGGCAAGTACTATTCCTACTATTGCGACGAGCACTTTGAGA
CACCATCCGGCTCTTACTGGGACCACATCCACTGTACCCAGGATGGCTGG
TCTCCCGCCGTGCCTTGCCTGAGAAAGTGTTACTTCCCCTATCTGGAGAA
CGGCTACAACCAGAATTATGGCAGGAAGTTTGTGCAGGGCAAGTCCATCG
ACGTGGCATGCCACCCAGGATACGCACTGCCTAAGGCACAGACCACAGTG
ACCTGTATGGAGAATGGCTGGAGCCCAACACCCAGGTGCATCCGCGTGAA
GACCTGTTCCAAGAGCAGCATCGACATCGAGAACGGCTTCATCAGCGAGT
CCCAGTACACCTATGCCCTGAAGGAGAAGGCCAAGTATCAGTGCAAGCTG
GGATACGTGACCGCAGACGGAGAAACATCTGGCAGCATCACCTGCGGCAA
GGATGGCTGGTCTGCCCAGCCCACATGCATCAAGAGCATCAAGACCGACT
GTCTGTCTCTGCCCAGCTTTGAGAATGCCATCCCTATGGGCGAGAAGAAG
GACGTGTACAAGGCCGGCGAGCAGGTGACATACACCTGCGCCACATACTA
TAAGATGGACGGCGCCAGCAACGTGACCTGTATCAATTCCCGGTGGACAG
GCAGACCTACCTGCAGGGATACAAGCTGCGTGAACCCACCCACCGTGCAG
AATGCCTATATCGTGTCCCGCCAGATGTCTAAGTACCCTAGCGGCGAGCG
GGTGAGATATCAGTGCCGGTCCCCATACGAGATGTTCGGCGACGAGGAAG
TGATGTGCCTGAACGGCAATTGGACAGAGCCTCCACAGTGCAAGGATAGC
ACCGGCAAGTGTGGACCACCTCCACCAATCGACAACGGCGATATCACATC
CTTTCCACTGAGCGTGTACGCCCCCGCCTCCTCTGTGGAGTATCAGTGCC
AGAACCTGTACCAGCTGGAGGGCAATAAGCGCATCACCTGTCGGAACGGC
CAGTGGTCTGAGCCTCCAAAGTGCCTGCACCCTTGCGTGATCTCCAGAGA
GATCATGGAGAACTATAATATCGCCCTGCGCTGGACAGCCAAGCAGAAGC
TGTACTCTCGGACCGGCGAGAGCGTGGAGTTCGTGTGCAAGCGGGGCTAT
AGACTGAGCTCCCGCAGCCACACACTGCGGACCACATGCTGGGACGGCAA
GCTGGAGTACCCAACCTGTGCCAAGAGGTGAGCCACGCGTAACACGTGCA
TGCGAGAGATCTGCGGCCGCGAGCTCGGGGATCCAGACATGATAAGATAC
ATTGATGAGTTTGGACAAACCACAACTAGAATGCAGTGAAAAAAATGCTT
TATTTGTGAAATTTGTGATGCTATTGCTTTATTTGTAACCATTATAAGCT
GCAATAAACAAGTTAACAACAACAATTGCATTCATTTTATGTTTCAGGTT
CAGGGGGAGGTGTGGGAGGTTTTTTAAAGCAAGTAAAACCTCTACAAATG
TGGTATGGCTGATTATGATCAATGCATCCTAGCCGGAGGAACCCCTAGTG
ATGGAGTTGGCCACTCCCTCTCTGCGCGCTCGCTCGCTCACTGAGGCCGC
CCGGGCAAAGCCCGGGCGTCGGGCGACCTTTGGTCGCCCGGCCTCAGTGA
GCGAGCGAGCGCGCAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 11)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTATCGA
TTGAACCAGGCGCGCCCAGTTTAAACAGTCCGGAACTAGTTATTAATAGT
AATCAATTACGGGGTCATTAGTTCATAGCCCATATATGGAGTTCCGCGTT
ACATAACTTACGGTAAATGGCCCGCCTGGCTGACCGCCCAACGACCCCCG
CCCATTGACGTCAATAATGACGTATGTTCCCATAGTAACGCCAATAGGGA
CTTTCCATTGACGTCAATGGGTGGAGTATTTACGGTAAACTGCCCACTTG
GCAGTACATCAAGTGTATCATATGCCAAGTACGCCCCCTATTGACGTCAA
TGACGGTAAATGGCCCGCCTGGCATTATGCCCAGTACATGACCTTATGGG
ACTTTCCTACTTGGCAGTACATCTACGTATTAGTCATCGCTATTACCATG
GTCGAGGTGAGCCCCACGTTCTGCTTCACTCTCCCCATCTCCCCCCCCTC
CCCACCCCCAATTTTGTATTTATTTATTTTTTAATTATTTTGTGCAGCGA
TGGGGGCGGGGGGGGGGGGGGGGCGCGCGCCAGGCGGGGCGGGGCGGGGC
GAGGGGCGGGGCGGGGCGAGGCGGAGAGGTGCGGCGGCAGCCAATCAGAG
CGGCGCGCTCCGAAAGTTTCCTTTTATGGCGAGGCGGCGGCGGCGGCGGC
CCTATAAAAAGCGAAGCGCGCGGCGGGCGGGGAGTCGCTGCGACGCTGCC
TTCGCCCCGTGCCCCGCTCCGCCGCCGCCTCGCGCCGCCCGCCCCGGCTC
TGACTGACCGCGTTACTCCCACAGGTGAGCGGGCGGGACGGCCCTTCTCC
TCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGG
AGCGGCTCGGGGGGTGCGTGCGTGTGTGTGTGCGTGGGGAGCGCCGCGTG
CGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCC
CCGCGGTGCGGGGGGGGCTGCGAGGGGAACAAAGGCTGCGTGCGGGGTGT
GTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGT
GCGGGGCTCCGTACGGGGCGTGGCGCGGGGCTCGCCGTGCCGGGCGGGGG
GTGGCGGCAGGTGGGGGTGCCGGGCGGGGCGGGGCCGCCTCGGGCCGGGG
AGGGCTCGGGGGAGGGGCGCGGCGGCCCCCGGAGCGCCGGCGGCTGTCGA
GGCGCGGCGAGCCGCAGCCATTGCCTTTTATGGTAATCGTGCGAGAGGGC
GCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCC
CCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGTC
TAGAGTCGACGGTCACCGGTCAGCTAGCACGCCACCATGCGCCTGCTGGC
CAAGATCATCTGCCTGATGCTGTGGGCCATCTGCGTGGCCGAGGACTGTA
ACGAGCTGCCCCCTCGGAGAAATACAGAGATCCTGACCGGCTCTTGGAGC
GATCAGACCTACCCAGAGGGCACACAGGCCATCTACAAGTGCAGGCCCGG
CTATCGCTCCCTGGGCAATATCATCATGGTGTGCAGGAAGGGAGAGTGGG
TGGCCCTGAACCCACTGAGAAAGTGCCAGAAGAGGCCATGTGGACACCCA
GGCGACACACCTTTCGGCACCTTTACACTGACCGGCGGCAACGTGTTCGA
GTACGGCGTGAAGGCCGTGTATACCTGCAATGAGGGCTACCAGCTGCTGG
GCGAGATCAACTACCGGGAGTGTGACACAGATGGCTGGACCAATGATATC
CCCATCTGCGAGGTGGTGAAGTGTCTGCCAGTGACCGCCCCCGAGAACGG
CAAGATCGTGAGCTCCGCCATGGAGCCTGACAGAGAGTATCACTTCGGCC
AGGCCGTGAGGTTCGTGTGCAATAGCGGCTACAAGATCGAGGGCGATGAG
GAGATGCACTGTTCCGACGATGGCTTCTGGTCTAAGGAGAAGCCCAAGTG
CGTGGAGATCTCCTGTAAGTCTCCTGACGTGATTAACGGCAGCCCAATCT
CCCAGAAGATCATCTATAAGGAGAATGAGCGGTTTCAGTACAAGTGCAAC
ATGGGCTACGAGTATTCCGAGAGAGGCGATGCCGTGTGCACAGAGTCTGG
ATGGAGGCCCCTGCCTAGCTGCGAGGAGAAGTCCACCCTGAAGCCTTGTG
ACTATCCAGATATCAAGCACGGCGGCCTGTATCACGAGAATATGAGGCGC
CCTTACTTCCCAGTGGCCGTGGGCAAGTACTATTCCTACTATTGCGACGA
GCACTTTGAGACACCATCCGGCTCTTACTGGGACCACATCCACTGTACCC
AGGATGGCTGGTCTCCCGCCGTGCCTTGCCTGAGAAAGTGTTACTTCCCC
TATCTGGAGAACGGCTACAACCAGAATTATGGCAGGAAGTTTGTGCAGGG
CAAGTCCATCGACGTGGCATGCCACCCAGGATACGCACTGCCTAAGGCAC
AGACCACAGTGACCTGTATGGAGAATGGCTGGAGCCCAACACCCAGGTGC
ATCCGCGTGAAGACCTGTTCCAAGAGCAGCATCGACATCGAGAACGGCTT
CATCAGCGAGTCCCAGTACACCTATGCCCTGAAGGAGAAGGCCAAGTATC
AGTGCAAGCTGGGATACGTGACCGCAGACGGAGAAACATCTGGCAGCATC
ACCTGCGGCAAGGATGGCTGGTCTGCCCAGCCCACATGCATCAAGAGCAT
CAAGACCGACTGTCTGTCTCTGCCCAGCTTTGAGAATGCCATCCCTATGG
GCGAGAAGAAGGACGTGTACAAGGCCGGCGAGCAGGTGACATACACCTGC
GCCACATACTATAAGATGGACGGCGCCAGCAACGTGACCTGTATCAATTC
CCGGTGGACAGGCAGACCTACCTGCAGGGATACAAGCTGCGTGAACCCAC
CCACCGTGCAGAATGCCTATATCGTGTCCCGCCAGATGTCTAAGTACCCT
AGCGGCGAGCGGGTGAGATATCAGTGCCGGTCCCCATACGAGATGTTCGG
CGACGAGGAAGTGATGTGCCTGAACGGCAATTGGACAGAGCCTCCACAGT
GCAAGGATAGCACCGGCAAGTGTGGACCACCTCCACCAATCGACAACGGC
GATATCACATCCTTTCCACTGAGCGTGTACGCCCCCGCCTCCTCTGTGGA
GTATCAGTGCCAGAACCTGTACCAGCTGGAGGGCAATAAGCGCATCACCT
GTCGGAACGGCCAGTGGTCTGAGCCTCCAAAGTGCCTGCACCCTTGCGTG
ATCTCCAGAGAGATCATGGAGAACTATAATATCGCCCTGCGCTGGACAGC
CAAGCAGAAGCTGTACTCTCGGACCGGCGAGAGCGTGGAGTTCGTGTGCA
AGCGGGGCTATAGACTGAGCTCCCGCAGCCACACACTGCGGACCACATGC
TGGGACGGCAAGCTGGAGTACCCAACCTGTGCCAAGAGGTGAGCCTAGGA
CTGTACAGTACGCGTAACACGTGGGGGATCCAGACATGATAAGATACATT
GATGAGTTTGGACAAACCACAACTAGAATGCAGTGAAAAAAATGCTTTAT
TTGTGAAATTTGTGATGCTATTGCTTTATTTGTAACCATTATAAGCTGCA
ATAAACAAGTTAACAACAACAATTGCATTCATTTTATGTTTCAGGTTCAG
GGGGAGGTGTGGGAGGTTTTTTAAAGCAAGTAAAACCTCTACAAATGTGG
TATGGCTGATTATGATCAATGCATGGTCCCAGCCTTCATTGAGCCTATTG
GTCCCCCCTCAGAACCCACCCACCTGGCAGTAGAGGATGTCTCTTGAGAC
ACCACTGTCTCCCTCAAGTTCAGGAGGCCCCCATTAATTAAGAGAGAGTG
GGAGCAGGAGGCCTGTCAGATGGCTACTCTTAAGTGGAGTACTGCCCAGA
GGGCTGCTCAGAGTGGGTGGCTGTAACCCTGCAGGGGCTGACAGAGCACA
CATCCATACTGGTGAAGGACCTGTGACCCACTGGCCAGACTGGCCGGCCG
GAGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 12)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTATCGA
TTGAACCAGGCGCGCCCAGTTTAAACAGTCCGGAACTAGTTATTAATAGT
AATCAATTACGGGGTCATTAGTTCATAGCCCATATATGGAGTTCCGCGTT
ACATAACTTACGGTAAATGGCCCGCCTGGCTGACCGCCCAACGACCCCCG
CCCATTGACGTCAATAATGACGTATGTTCCCATAGTAACGCCAATAGGGA
CTTTCCATTGACGTCAATGGGTGGAGTATTTACGGTAAACTGCCCACTTG
GCAGTACATCAAGTGTATCATATGCCAAGTACGCCCCCTATTGACGTCAA
TGACGGTAAATGGCCCGCCTGGCATTATGCCCAGTACATGACCTTATGGG
ACTTTCCTACTTGGCAGTACATCTACGTATTAGTCATCGCTATTACCATG
GTCGAGGTGAGCCCCACGTTCTGCTTCACTCTCCCCATCTCCCCCCCCTC
CCCACCCCCAATTTTGTATTTATTTATTTTTTAATTATTTTGTGCAGCGA
TGGGGGCGGGGGGGGGGGGGGGGCGCGCGCCAGGCGGGGCGGGGCGGGGC
GAGGGGCGGGGCGGGGCGAGGCGGAGAGGTGCGGCGGCAGCCAATCAGAG
CGGCGCGCTCCGAAAGTTTCCTTTTATGGCGAGGCGGCGGCGGCGGCGGC
CCTATAAAAAGCGAAGCGCGCGGCGGGCGGGGAGTCGCTGCGACGCTGCC
TTCGCCCCGTGCCCCGCTCCGCCGCCGCCTCGCGCCGCCCGCCCCGGCTC
TGACTGACCGCGTTACTCCCACAGGTGAGCGGGCGGGACGGCCCTTCTCC
TCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGG
AGCGGCTCGGGGGGTGCGTGCGTGTGTGTGTGCGTGGGGAGCGCCGCGTG
CGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCC
CCGCGGTGCGGGGGGGGCTGCGAGGGGAACAAAGGCTGCGTGCGGGGTGT
GTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGT
GCGGGGCTCCGTACGGGGCGTGGCGCGGGGCTCGCCGTGCCGGGCGGGGG
GTGGCGGCAGGTGGGGGTGCCGGGCGGGGCGGGGCCGCCTCGGGCCGGGG
AGGGCTCGGGGGAGGGGCGCGGCGGCCCCCGGAGCGCCGGCGGCTGTCGA
GGCGCGGCGAGCCGCAGCCATTGCCTTTTATGGTAATCGTGCGAGAGGGC
GCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGC
AGGAAGGAAATGGGCGGGGAGGGCCTTCGTGCGTCGCCGCGCCGCCGTCC
CCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGTC
TAGAGTCGACGGTCACCGGTCAGCTAGCACGCCACCATGAGGCTGCTGGC
CAAGATCATCTGCCTGATGCTGTGGGCCATCTGTGTGGCTGAGGACTGTA
ATGAGCTGCCCCCTAGGAGAAATACAGAGATCCTGACTGGCTCTTGGTCT
GATCAGACCTACCCAGAGGGCACACAGGCCATCTACAAGTGCAGGCCTGG
CTATAGATCCCTGGGCAATATCATCATGGTGTGCAGGAAGGGAGAGTGGG
TGGCCCTGAACCCACTGAGAAAGTGCCAGAAGAGGCCATGTGGACACCCA
GGAGACACACCTTTTGGCACCTTTACACTGACAGGGGGCAATGTGTTTGA
GTATGGTGTGAAGGCAGTGTATACCTGCAATGAGGGCTACCAGCTGCTGG
GAGAGATCAACTACAGGGAGTGTGACACAGATGGCTGGACCAATGATATC
CCCATCTGTGAGGTGGTGAAGTGTCTGCCAGTGACTGCCCCTGAGAATGG
CAAGATAGTGAGCTCTGCCATGGAGCCTGACAGAGAGTATCACTTTGGCC
AGGCTGTGAGGTTTGTGTGCAATAGTGGCTACAAGATTGAGGGGGATGAG
GAGATGCACTGTTCAGATGATGGCTTCTGGTCTAAGGAGAAGCCCAAGTG
TGTGGAGATCTCCTGTAAGTCTCCTGATGTGATTAATGGCAGCCCAATCT
CCCAGAAGATCATCTATAAGGAGAATGAGAGATTTCAGTACAAGTGCAAC
ATGGGCTATGAGTATTCAGAGAGAGGTGATGCTGTGTGCACAGAGTCTGG
ATGGAGGCCCCTGCCTAGCTGTGAGGAGAAGTCCACCCTGAAGCCTTGTG
ACTATCCAGATATCAAGCATGGAGGCCTGTATCATGAGAATATGAGGAGG
CCTTACTTCCCAGTGGCTGTGGGCAAGTACTATTCCTACTATTGTGATGA
GCACTTTGAGACACCATCTGGCTCTTACTGGGACCACATCCACTGTACCC
AGGATGGCTGGTCTCCTGCTGTGCCTTGCCTGAGAAAGTGTTACTTCCCC
TATCTGGAGAATGGCTACAACCAGAATTATGGCAGGAAGTTTGTGCAGGG
CAAGTCCATTGATGTGGCATGCCACCCAGGATATGCACTGCCTAAGGCAC
AGACCACAGTGACCTGTATGGAGAATGGCTGGAGCCCAACACCCAGGTGC
ATCAGGGTGAAGACCTGTTCCAAGAGCAGCATTGACATAGAGAATGGCTT
CATCTCAGAGTCCCAGTACACCTATGCCCTGAAGGAGAAGGCCAAGTATC
AGTGCAAGCTGGGATATGTGACAGCAGATGGAGAAACATCTGGCAGCATC
ACCTGTGGCAAGGATGGCTGGTCTGCCCAGCCCACATGCATCAAGAGCAT
CAAGACTGACTGTCTGTCTCTGCCCAGCTTTGAGAATGCCATCCCTATGG
GTGAGAAGAAGGATGTGTACAAGGCTGGAGAGCAGGTGACATACACCTGT
GCCACATACTATAAGATGGATGGAGCCAGCAATGTGACCTGTATCAATTC
CAGATGGACAGGCAGACCTACCTGCAGGGATACAAGCTGTGTGAACCCAC
CCACTGTGCAGAATGCCTATATTGTGTCCAGGCAGATGTCTAAGTACCCT
TCTGGAGAGAGAGTGAGATATCAGTGCAGATCCCCATATGAGATGTTTGG
GGATGAGGAAGTGATGTGCCTGAATGGCAATTGGACAGAGCCTCCACAGT
GCAAGGATAGCACTGGCAAGTGTGGACCACCTCCACCAATTGACAATGGA
GATATCACATCCTTTCCACTGTCAGTGTATGCCCCTGCCTCCTCTGTGGA
GTATCAGTGCCAGAACCTGTACCAGCTGGAGGGCAATAAGAGAATCACCT
GTAGAAATGGCCAGTGGTCTGAGCCTCCAAAGTGCCTGCACCCTTGTGTG
ATCTCCAGAGAGATCATGGAGAACTATAATATAGCCCTGAGGTGGACAGC
CAAGCAGAAGCTGTACTCTAGGACTGGGGAGTCAGTGGAGTTTGTGTGCA
AGAGAGGCTATAGACTGAGCTCCAGAAGCCACACACTGAGGACCACATGC
TGGGATGGCAAGCTGGAGTACCCAACCTGTGCCAAGAGGTGAGCCTAGGA
CTGTACAGTACGCGTAACACGTGGGGGATCCAGACATGATAAGATACATT
GATGAGTTTGGACAAACCACAACTAGAATGCAGTGAAAAAAATGCTTTAT
TTGTGAAATTTGTGATGCTATTGCTTTATTTGTAACCATTATAAGCTGCA
ATAAACAAGTTAACAACAACAATTGCATTCATTTTATGTTTCAGGTTCAG
GGGGAGGTGTGGGAGGTTTTTTAAAGCAAGTAAAACCTCTACAAATGTGG
TATGGCTGATTATGATCAATGCATGGTCCCAGCCTTCATTGAGCCTATTG
GTCCCCCCTCAGAACCCACCCACCTGGCAGTAGAGGATGTCTCTTGAGAC
ACCACTGTCTCCCTCAAGTTCAGGAGGCCCCCATTAATTAAGAGAGAGTG
GGAGCAGGAGGCCTGTCAGATGGCTACTCTTAAGTGGAGTACTGCCCAGA
GGGCTGCTCAGAGTGGGTGGCTGTAACCCTGCAGGGGCTGACAGAGCACA
CATCCATACTGGTGAAGGACCTGTGACCCACTGGCCAGACTGGCCGGCCG
GAGGAACCCCTAGTGATGGAGTTGGCCACTCCCTCTCTGCGCGCTCGCTC
GCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAA

In related aspects, the heterologous nucleic acid comprises the following nucleotide sequence or a nucleotide sequence at least 80%, at least 85%, at least 90%, at least 95%, at least 98% or at least 99% identical thereto:

(SEQ ID NO: 13)
TTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGC
GAGCGCGCAGAGAGGGAGTGGCCAACTCCATCACTAGGGGTTCCTATCGA
TTGAACCAGGCGCGCCCAGTTTAAACAGTCCGGAACTAGTTATTAATAGT
AATCAATTACGGGGTCATTAGTTCATAGCCCATATATGGAGTTCCGCGTT
ACATAACTTACGGTAAATGGCCCGCCTGGCTGACCGCCCAACGACCCCCG
CCCATTGACGTCAATAATGACGTATGTTCCCATAGTAACGCCAATAGGGA
CTTTCCATTGACGTCAATGGGTGGAGTATTTACGGTAAACTGCCCACTTG
GCAGTACATCAAGTGTATCATATGCCAAGTACGCCCCCTATTGACGTCAA
TGACGGTAAATGGCCCGCCTGGCATTATGCCCAGTACATGACCTTATGGG
ACTTTCCTACTTGGCAGTACATCTACGTATTAGTCATCGCTATTACCATG
GTCGAGGTGAGCCCCACGTTCTGCTTCACTCTCCCCATCTCCCCCCCCTC
CCCACCCCCAATTTTGTATTTATTTATTTTTTAATTATTTTGTGCAGCGA
TGGGGGCGGGGGGGGGGGGGGGGCGCGCGCCAGGCGGGGCGGGGGGGGCG
AGGGGCGGGGCGGGGCGAGGCGGAGAGGTGCGGCGGCAGCCAATCAGAGC
GGCGCGCTCCGAAAGTTTCCTTTTATGGCGAGGCGGCGGCGGCGGCGGCC
CTATAAAAAGCGAAGCGCGCGGCGGGCGGGGAGTCGCTGCGACGCTGCCT
TCGCCCCGTGCCCCGCTCCGCCGCCGCCTCGCGCCGCCCGCCCCGGCTCT
GACTGACCGCGTTACTCCCACAGGTGAGCGGGCGGGACGGCCCTTCTCCT
CCGGGCTGTAATTAGCGCTTGGTTTAATGACGGCTTGTTTCTTTTCTGTG
GCTGCGTGAAAGCCTTGAGGGGCTCCGGGAGGGCCCTTTGTGCGGGGGGA
GCGGCTCGGGGGGTGCGTGCGTGTGTGTGTGCGTGGGGAGCGCCGCGTGC
GGCTCCGCGCTGCCCGGCGGCTGTGAGCGCTGCGGGCGCGGCGCGGGGCT
TTGTGCGCTCCGCAGTGTGCGCGAGGGGAGCGCGGCCGGGGGCGGTGCCC
CGCGGTGCGGGGGGGGCTGCGAGGGGAACAAAGGCTGCGTGCGGGGTGTG
TGCGTGGGGGGGTGAGCAGGGGGTGTGGGCGCGTCGGTCGGGCTGCAACC
CCCCCTGCACCCCCCTCCCCGAGTTGCTGAGCACGGCCCGGCTTCGGGTG
CGGGGCTCCGTACGGGGCGTGGCGCGGGGCTCGCCGTGCCGGGCGGGGGG
TGGCGGCAGGTGGGGGTGCCGGGCGGGGCGGGGCCGCCTCGGGCCGGGGA
GGGCTCGGGGGAGGGGCGCGGCGGCCCCCGGAGCGCCGGCGGCTGTCGAG
GCGCGGCGAGCCGCAGCCATTGCCTTTTATGGTAATCGTGCGAGAGGGCG
CAGGGACTTCCTTTGTCCCAAATCTGTGCGGAGCCGAAATCTGGGAGGCG
CCGCCGCACCCCCTCTAGCGGGCGCGGGGCGAAGCGGTGCGGCGCCGGCA
GGAAGGAAATGGGCGGGGAGGGCCTTCGTGCGTCGCCGCGCCGCCGTCCC
CTTCTCCCTCTCCAGCCTCGGGGCTGTCCGCGGGGGGACGGCTGCCTTCG
GGGGGGACGGGGCAGGGCGGGGTTCGGCTTCTGGCGTGTGACCGGCGGCT
CTAGAGCCTCTGCTAACCATGTTCATGCCTTCTTCTTTTTCCTACAGTCT
AGAGTCGACGGTCACCGGTCAGCTAGCACGCCACCATGAGACTTCTAGCA
AAGATTATTTGCCTTATGTTATGGGCTATTTGTGTAGCAGAAGATTGCAA
TGAACTTCCTCCAAGAAGAAATACAGAAATTCTGACAGGTTCCTGGTCTG
ACCAAACATATCCAGAAGGCACCCAGGCTATCTATAAATGCAGGCCTGGA
TATAGATCTCTTGGAAATATCATAATGGTATGCAGGAAGGGAGAATGGGT
TGCTCTTAATCCATTAAGGAAATGTCAGAAAAGGCCCTGTGGACATCCTG
GAGATACTCCTTTTGGTACTTTTACCCTTACAGGAGGAAATGTGTTTGAA
TATGGTGTAAAAGCTGTGTATACATGTAATGAGGGGTATCAATTGCTAGG
TGAGATTAATTACAGGGAATGTGACACAGATGGATGGACCAATGATATTC
CTATATGTGAAGTTGTGAAGTGTTTACCAGTGACAGCACCAGAGAATGGA
AAAATTGTCAGTAGTGCAATGGAACCAGATAGAGAATACCATTTTGGACA
AGCAGTAAGATTTGTATGTAACTCAGGCTACAAGATTGAAGGAGATGAAG
AAATGCATTGTTCAGATGATGGTTTTTGGAGTAAAGAGAAACCAAAGTGT
GTGGAAATTTCATGCAAATCCCCAGATGTTATAAATGGATCTCCTATATC
TCAGAAGATTATTTATAAGGAGAATGAAAGATTTCAATATAAATGTAACA
TGGGTTATGAATACAGTGAAAGAGGAGATGCTGTATGCACTGAATCTGGA
TGGAGACCTTTGCCTTCATGTGAAGAAAAATCAACCTTGAAACCTTGTGA
TTATCCAGACATTAAACATGGAGGTCTATATCATGAGAATATGAGAAGAC
CATACTTTCCAGTAGCTGTAGGAAAATATTACTCCTATTACTGTGATGAA
CATTTTGAGACTCCTTCAGGAAGTTACTGGGATCACATTCATTGCACACA
AGATGGATGGAGTCCAGCAGTACCATGCCTCAGAAAATGTTATTTTCCTT
ATTTGGAAAATGGATATAATCAAAATTATGGAAGAAAGTTTGTACAGGGT
AAATCTATAGATGTTGCCTGCCATCCTGGCTATGCTCTTCCAAAAGCTCA
GACCACAGTTACATGTATGGAGAATGGCTGGTCTCCTACTCCCAGATGCA
TCAGAGTCAAAACATGTTCCAAATCAAGTATAGATATTGAGAATGGGTTT
ATTTCTGAATCTCAGTATACATATGCCTTAAAAGAAAAAGCTAAATATCA
ATGCAAACTAGGATATGTAACAGCAGATGGTGAAACATCAGGATCAATTA
CATGTGGGAAAGATGGATGGTCAGCTCAACCCACCTGCATTAAATCTATA
AAAACAGATTGTCTCAGTTTACCTAGCTTTGAAAATGCCATACCCATGGG
AGAGAAGAAGGATGTGTATAAGGCTGGTGAGCAAGTGACTTACACTTGTG
CAACATATTACAAAATGGATGGAGCCAGTAATGTAACATGCATTAATAGC
AGATGGACAGGAAGGCCAACATGCAGAGACACCTCCTGTGTGAATCCTCC
CACAGTACAAAATGCTTATATAGTGAGTAGACAGATGAGTAAATATCCAT
CTGGTGAGAGAGTAAGATATCAATGTAGGAGCCCTTATGAAATGTTTGGG
GATGAAGAAGTGATGTGTTTAAATGGAAACTGGACAGAACCACCTCAATG
CAAAGATTCTACAGGAAAATGTGGGCCCCCTCCACCTATTGACAATGGGG
ACATTACTTCATTCCCCTTGTCAGTATATGCTCCAGCTTCATCAGTTGAG
TACCAATGCCAGAACTTGTATCAACTTGAGGGTAACAAGAGGATAACATG
TAGAAATGGACAATGGTCAGAACCACCAAAATGCTTACATCCCTGTGTAA
TATCCAGGGAAATTATGGAAAATTATAACATAGCATTAAGGTGGACAGCC
AAACAGAAGCTTTATTCTAGAACAGGTGAATCAGTTGAATTTGTGTGTAA
AAGAGGATATAGACTTTCATCAAGGTCTCACACATTGAGGACAACATGTT
GGGATGGGAAACTGGAGTATCCAACTTGTGCAAAAAGATGAGCCTAGGAC
TGTACAGTACGCGTAACACGTGGGGGATCCAGACATGATAAGATACATTG
ATGAGTTTGGACAAACCACAACTAGAATGCAGTGAAAAAAATGCTTTATT
TGTGAAATTTGTGATGCTATTGCTTTATTTGTAACCATTATAAGCTGCAA
TAAACAAGTTAACAACAACAATTGCATTCATTTTATGTTTCAGGTTCAGG
GGGAGGTGTGGGAGGTTTTTTAAAGCAAGTAAAACCTCTACAAATGTGGT
ATGGCTGATTATGATCAATGCATGGTCCCAGCCTTCATTGAGCCTATTGG
TCCCCCCTCAGAACCCACCCACCTGGCAGTAGAGGATGTCTCTTGAGACA
CCACTGTCTCCCTCAAGTTCAGGAGGCCCCCATTAATTAAGAGAGAGTGG
GAGCAGGAGGCCTGTCAGATGGCTACTCTTAAGTGGAGTACTGCCCAGAG
GGCTGCTCAGAGTGGGTGGCTGTAACCCTGCAGGGGCTGACAGAGCACAC
ATCCATACTGGTGAAGGACCTGTGACCCACTGGCCAGACTGGCCGGCCGG
AGGAACCCCTAGTGATGGAGTTGGCCACTCCCTCTCTGCGCGCTCGCTCG
CTCACTGAGGCCGCCCGGGCAAAGCCCGGGCGTCGGGCGACCTTTGGTCG
CCCGGCCTCAGTGAGCGAGCGAGCGCGCAGAGAGGGAGTGGCCAA

In some embodiments, the pharmaceutical composition comprises 1×10⁸ to 1×10¹⁵ vector particles (vp) or vector genomes (vg), 1×10¹⁰ to 1×10¹³ vector particles or vector genomes, or about 1×10¹⁰, about 2×10¹⁰, 3×10¹⁰, about 4×10¹⁰, about 5×10¹⁰, about 6×10¹⁰, about 7×10¹⁰, about 8×10¹⁰, about 9×10¹⁰, about 1×10¹¹, about 2×10¹¹, about 3×10¹¹, about 4×10¹¹, about 5×10¹¹, about 6×10¹¹, about 7×10¹¹, about 8×10¹¹, about 9×10¹¹, about 1×10¹², about 2×10¹², about 3×10¹², about 4×10¹², about 5×10¹², about 6×10¹², about 7×10¹², about 8×10¹², about 9×10¹² or about 1×10¹³ vector particles or vector genomes. In some aspects, the pharmaceutical composition comprises about 1×10¹¹ to about 1×10¹² vector particles or vector genomes.

In some aspects, the pharmaceutical composition maintains a relative potency in liquid form of at least 90% over a period of at least 2 weeks, at least 3 weeks, at least 4 weeks, at least 5 weeks, at least 6 weeks, at least 7 weeks, at least 8 weeks, at least 9 weeks, at least 10 weeks, at least 3 months, at least 4 months, at least 5 months or at least 6 months at 25° C. and 60% relative humidity.

In exemplary aspects, the composition comprising AAV is storage stable as a liquid for at least 1 month, at least 2 months, at least 3 months, at least 4 months, at least 5 months, at least 6 months, at least 7 months, at least 8 months, at least 9 months, at least 10 months, at least 11 months, or at least 12 months. In exemplary aspects, greater than 80% of the initial amount of AAV (e.g., the amount of AAV in the composition prior to storage) is potent after the storage period (e.g., a storage period of about 1 month, about 2 months, about 3 months, about 4 months, about 5 months, or about 6 months or longer). In exemplary aspects, greater than 90% of the initial amount of AAV is potent after the storage period (e.g., a storage period of about 1 month, about 2 months, 3 months, about 4 months, about 5 months, or about 6 months or longer). In exemplary aspects, greater than 95% of the initial amount of AAV is potent after the storage period (e.g., a storage period of about 3 months, about 4 months, about 5 months, or about 6 months or longer). In exemplary aspects, the biopotency of the AAV at the end of the storage period is substantially the same as the biopotency of the AAV at the beginning of the storage period. In exemplary aspects, the biopotency of the AAV at the end of the storage period is increased relative to the biopotency of the AAV at the beginning of the storage period. In exemplary aspects, the appearance of the composition at the end of the storage period is substantially the same as the composition at the beginning of the storage period. In exemplary aspects, the appearance of the composition at the end of the storage period is characterized by having no visible particles. In exemplary aspects, the particle concentration of the composition at the end of the storage period is substantially the same as the particle concentration of the composition at the beginning of the storage period. In exemplary aspects, the particle concentration of the composition at the end of the storage period is determined by microflow imaging (MFI).

In some embodiments, the pharmaceutical composition is maintained at about −60° C. or below for a substantial portion (e.g., the duration) of the storage period. In other embodiments, the pharmaceutical composition is maintained at from about 2° C. to about 8° C. for a substantial portion (e.g., the duration) of the storage period.

In some embodiments, a method for treating a disorder in a subject in need thereof is provided, the method comprising administering to the subject a pharmaceutical composition as described herein in an amount effective to treat the disorder. In some preferred embodiments, the pharmaceutical composition is administered intraocularly to a human with a VEGF-related ocular disorder in an amount effective to treat the VEGF-related ocular disorder, preferably wherein the pharmaceutical composition is administered via intravitreal, subretinal and/or suprachoroidal injection, more preferably via a single intravitreal injection. In other embodiments, the disorder is selected from those listed at paragraphs 218 and 246 of US Patent Application Publication No. 2020/0282077.

The formulations disclosed herein may be formulated for administration via known methods, such as intraocular administration (e.g., via intravitreal, subretinal and/or suprachoroidal administration). In some preferred embodiments, the pharmaceutical composition is administered intraocularly to a human, preferably wherein the pharmaceutical composition is administered via intravitreal, subretinal and/or suprachoroidal injection, more preferably via a single intravitreal injection.

In some embodiments, the treatment regimen includes administering one or more doses over an extended period of time. In certain cases, a single dose (e.g., a single dosage unit) is administered to the subject, and the initial dose may be followed by one or more doses administered to the subject at a subsequent time. In some instances, more than one dose (e.g., more than one dosage unit) is administered to the subject, and the initial doses may be followed by one or more doses administered to the subject at a subsequent time. For example, a single dose (e.g., a single dosage unit) may be administered to the subject, and the single dose may be followed by a single dose administered to the subject at a subsequent time. Additional single doses may be administered at subsequent points in time. In other cases, a single dose (e.g., a single dosage unit) may be administered to the subject, and the single dose may be followed by two doses administered to the subject at a subsequent time. Additional single or multiple doses may be administered at subsequent points in time.

In certain embodiments, dosage units of the present disclosure can be administered prior to, concurrent with, or subsequent to other active agents for treating related or unrelated conditions, e.g., in combination therapy. Administration of separate pharmaceutical compositions can be performed simultaneously or at different times (e.g., sequentially, in either order, on the same day, or on different days), as long as a therapeutically effective effect of the combination of these substances is caused in the subject undergoing therapy. Accordingly, aspects of the present disclosure further include combination therapies. In certain embodiments, the subject method includes administering a therapeutically effective amount of one or more additional active agents. By combination therapy is meant that a AAV composition (e.g., as described herein) can be used in a combination with another therapeutic agent to treat a single disease or condition. In certain embodiments, a compound of the present disclosure is administered concurrently with the administration of another therapeutic agent, which can be administered as a component of a composition including the compound of the present disclosure or as a component of a different composition. In certain embodiments, a composition including a compound of the present disclosure is administered prior or subsequent to administration of another therapeutic agent.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

The below sections summarize key findings that led to identification of an improved rAAV formulation (10 mM Tris buffer, pH 7.9, 180 mM NaCl, 0.005% Pluronic F68) which exhibits reduced risk of subvisible particle formation as well as improved liquid storage stability, which is consistent with the stabilization provided by increased ionic strength and reduced rAAV surface adsorption by use of surfactant and the importance of optimizing formulation pH. Wright, J F et al. Mol Ther. 2005 July; 12(1):171-8); Patricio M I, Mol Ther Methods Clin Dev. 2019 Nov. 20; 17:99-106; Srivastava, A. et al. Journal of Pharmaceutical Sciences, Volume 110, Issue 7, 2021. The trend of increasing stability of rAAV (comprising a capsid protein of SEQ ID NO:3 and a nucleic acid comprising the nucleotide sequence of SEQ ID NO:6), at higher pH initially drove the selection of the Tris buffer component (Tris Hydrochloride/Tromethamine) in the improved rAAV formulation. However, in at least one study Tris buffer was also shown to reduce susceptibility to chemical degradation (e.g., deamidation and oxidation) compared to Hepes and Phosphate formulations made at the same target pH and equivalent concentrations of sodium chloride and Pluronic F68.

TABLE 1
Improved rAAV Formulation Components and Functions

| Component | Function | Target Concentration |
|---|---|---|
| Pluronic ® F68 | Surfactant | 0.005% |
| Tris Hydrochloride | Buffering Agent | 6.45 mM |
| Tromethamine | Buffering Agent | 3.55 mM |
| Sodium Chloride | Tonicity | 180 mM |
| Water for Injection | Solvent | QS |

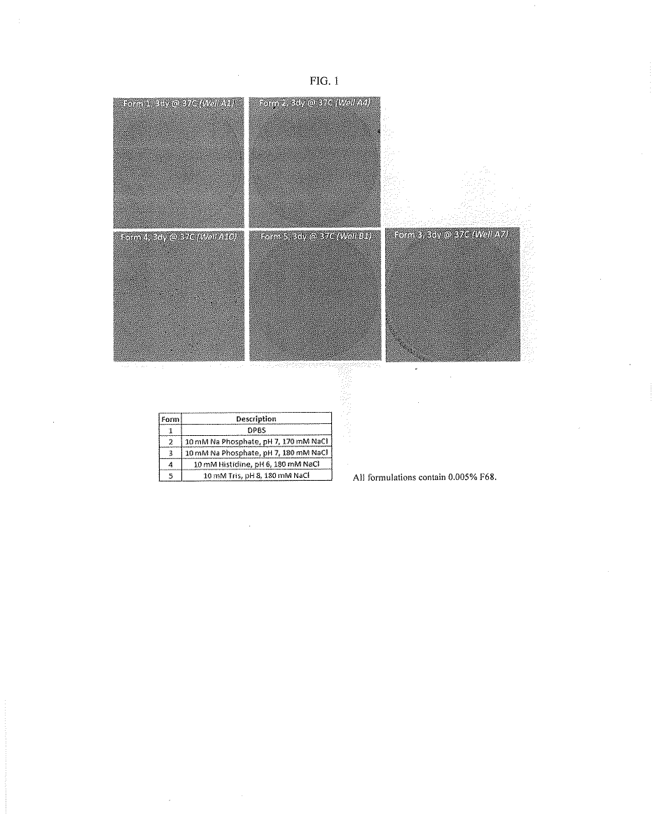

rAAV, comprising a capsid protein of SEQ ID NO:3 and a nucleic acid comprising the nucleotide sequence of SEQ ID NO:6, was buffer-exchanged into 5 different buffers (Form 1-5) and subjected to various forced degradation conditions. A summary of titer, DLS and subvisible particle testing results can be found in Table 2. Titers were similar for most conditions, except after 3-day storage at 37° C. Interestingly, 37° C. titer values were suggestive of increased thermal stability for Form 4 (10 mM Histidine, pH 6, 180 mM NaCl, 0.005% F68). However, Form 4 showed markedly decreased colloidal stability (i.e., increased poly-dispersity index (PDI) and decreased % area, 10-100 d·nm, via dynamic light scattering (DLS) and increased SVPs) at most conditions tested. Conversely, Form 2 (10 mM Sodium Phosphate, pH 7.0, 170 mM NaCl, 0.005% F68), Form 3 (10 mM Sodium Phosphate, pH 7.0, 180 mM NaCl, 0.005% F68) and Form 5 (10 mM Tris buffer, pH 8.0, 180 mM NaCl, 0.005% F68) showed improved colloidal stability, relative to the Form 1 control (DPBS, 0.005% F68), at most conditions. Representative backgrounded membrane images (FIG. 1) have been included for the 3-day storage condition to further illustrate the higher risk of subvisible particle formation exhibited by Form 1 and Form 4. These results suggested that pH 7 or pH 8 formulations containing increased sodium chloride were likely to result in an improvement over buffer (DPBS, 0.005% F68).

TABLE 2
Titer, DLS, and SVP Testing Results

| | | | | | | Subvisible Particle Testing Results⁴ | | | |
|---|---|---|---|---|---|---|---|---|---|
| | | | Titer | | % Area | Particles/mL | | | |
| Form | Description | Condition | (vg/mL)¹ | PDI² | (10-100 d · nm)³ | ≥2 μm | ≥10 μm | ≥25 μm | ≥50 μm |
| 1 | DPBS | T = 0 | 2.53E+13 | 0.054 | 100.0 | 6120 | 0 | 0 | 0 |
| 2 | 10 mM Na Phosphate, pH 7, 170 mM NaCl | T = 0 | 2.17E+13 | 0.012 | 100.0 | 280 | 13 | 0 | 0 |
| 3 | 10 mM Na Phosphate, pH 7, 120 mM NaCl | T = 0 | 2.27E+13 | 0.019 | 100.0 | 240 | 13 | 0 | 0 |
| 4 | 10 mM Histidine, pH 6, 180 mM NaCl | T = 0 | 2.43E+13 | 0.343 | 98.0 | 253 | 0 | 0 | 0 |
| 5 | 10 mM Tris, pH 8, 180 mM NaCl | T = 0 | 2.14E+13 | 0.020 | 100.0 | 360 | 27 | 0 | 0 |
| 1 | DPBS | 3 × FT | 2.51E+13 | 0.123 | 100.0 | 9720 | 67 | 40 | 13 |
| 2 | 10 mM Na Phosphate, pH 7, 170 mM NaCl | 3 × FT | 2.11E+13 | 0.015 | 100.0 | 453 | 27 | 0 | 0 |
| 3 | 10 mM Na Phosphate, pH 7, 180 mM NaCl | 3 × FT | 2.12E+13 | 0.009 | 100.0 | 480 | 53 | 0 | 0 |
| 4 | 10 mM Histidine, pH 6, 180 mM NaCl | 3 × FT | 2.29E+13 | 0.714 | 93.2 | 7267 | 40 | 13 | 13 |
| 5 | 10 mM Tris, pH 8, 180 mM NaCl | 3 × FT | 2.25E+13 | 0.023 | 100.0 | 333 | 27 | 0 | 0 |
| 1 | DPBS | 3 dy @ 37 C. | 2.21E+13 | 0.092 | 100.0 | 6400 | 1227 | 227 | 0 |
| 2 | 10 mM Na Phosphate, pH 7, 170 mM NaCl | 3 dy @ 37 C. | 1.91E+13 | 0.013 | 100.0 | 2280 | 107 | 13 | 0 |
| 3 | 10 mM Na Phosphate, pH 7, 180 mM NaCl | 3 dy @ 37 C. | 2.08E+13 | 0.021 | 100.0 | 1347 | 40 | 13 | 0 |
| 4 | 10 mM Histidine, pH 6, 180 mM NaCl | 3 dy @ 37 C. | 2.40E+13 | 0.332 | 98.9 | 132040 | 30120 | 3040 | 267 |
| 5 | 10 mM Tris, pH 8, 180 mM NaCl | 3 dy @ 37 C. | 1.70E+13 | 0.020 | 100.0 | 1387 | 67 | 13 | 13 |
| 1 | DPBS | 10 × FT | 2.55E+13 | 0.138 | 100.0 | 257373 | 9440 | 267 | 0 |
| 2 | 10 mM Na Phosphate, pH 7, 170 mM NaCl | 10 × FT | 2.14E+13 | 0.012 | 100.0 | 1280 | 67 | 27 | 27 |
| 3 | 10 mM Na Phosphate, pH 7, 180 mM NaCl | 10 × FT | 2.16E+13 | 0.014 | 100.0 | 1213 | 80 | 40 | 27 |
| 4 | 10 mM Histidine, pH 6, 180 mM NaCl | 10 × FT | 2.40E+13 | 1.368 | 80.8 | 1624680 | 16987 | 93 | 27 |
| 5 | 10 mM Tris, pH 8, 180 mM NaCl | 10 × FT | 2.03E+13 | 0.008 | 100.0 | 6747 | 213 | 40 | 0 |
| 1 | DPBS | 1 M @ RT | 2.17E+13 | 0.013 | 100.0 | 294560 | 340 | 100 | 40 |
| 2 | 10 mM Na Phosphate, pH 7, 170 mM NaCl | 1 M @ RT | 2.04E+13 | 0.004 | 100.0 | 173 | 27 | 13 | 13 |
| 3 | 10 mM Na Phosphate, pH 7, 180 mM NaCl | 1 M @ RT | 2.04E+13 | 0.017 | 100.0 | 253 | 40 | 13 | 13 |
| 4 | 10 mM Histidine, pH 6, 180 mM NaCl | 1 M @ RT | 2.30E+13 | 0.215 | 99.7 | 198213 | 15493 | 120 | 27 |
| 5 | 10 mM Tris, pH 8, 180 mM NaCl | 1 M @ RT | 2.03E+13 | 0.033 | 100.0 | 720 | 100 | 20 | 0 |

All formulations also contain 0.005% Pluronic F68
¹T = 0 values for Form 1 and Form 2 represent n = 2 averages. All other titer values are n = 1
²Polydispersity Index
³% Area by Volume
⁴Mean counts for n = 3 wells except for Form 1 and Form 5 after 1 M RT storage (n = 2 wells)

### pH Optimization

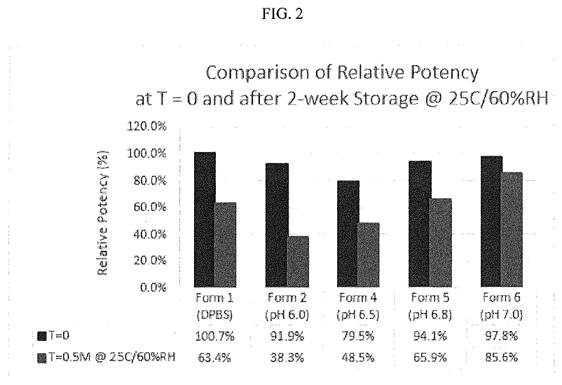

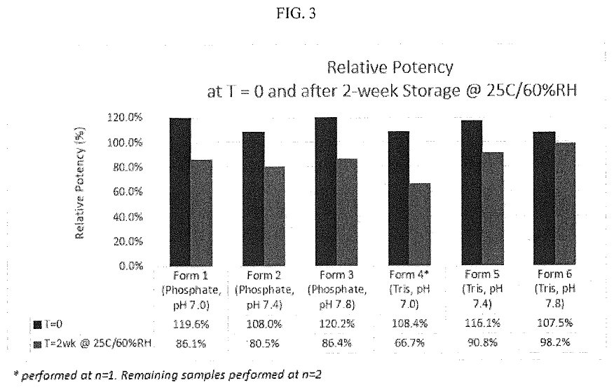

The next studies aimed to further assess pH dependence. In the first study, rAAV (comprising a capsid protein of SEQ ID NO:3 and a nucleic acid comprising the nucleotide sequence of SEQ TD NO:6) was buffer-exchanged into 6 different formulations containing Sodium Phosphate buffer ranging from pH 6.0 to 7.0. In the second study, rAAV (comprising a capsid protein of SEQ TD NO:3 and a nucleic acid comprising the nucleotide sequence of SEQ ID NO:6) was buffer-exchanged into Phosphate or Tris buffer at a pH 7.0, 7.4 and 7.8. These studies (along with subsequent Formulation Development experiments) included testing for aflibercept (AFLIB) expression using a modified form of the release assay tailored to high throughput sample analysis. Aflibercept expression was reported as relative potency (00 RP), or AFLTB expression relative to a standard. Like prior results, the first of these studies demonstrated decreased colloidal stability when pH was decreased from 7.0 to 6.0 (Table 3). Decreased relative potency (FIG. 2) was also observed for lower pH formulations in response to thermal stress. Conversely, a decreased risk of subvisible particle formation and equal or better 2-week storage stability at 25° C./60% RH was noted for the rAAV formulated in 10 mM Sodium Phosphate, pH 7.0, 175 mM NaCl, 0.005% F68 compared to the rAAV formulated in Form 1 (DPBS, 0.005% F68). The results of the second study (Table 4) demonstrated a further reduction in subvisible particle risk as pH was increased from 7.0 to 7.8. Most interesting was rAAV formulated in 10 mM Tris buffer, pH 7.8, 180 mM NaCl, 0.005% F68 which not only showed low subvisible particle counts for all conditions but also the highest relative potency (98.2%) after 2-week storage at 25° C./60% RH (FIG. 3).

TABLE 3
Study Results

| | | | | | Relative |
|---|---|---|---|---|---|
| | | | | Titer | Potency |
| Form | Description | Condition | pH | (vg/mL)¹ | % |
| 1 | DPBS | T = 0 | 7.00 | 2.06E+13 | 100.7% |
| 2 | 10 mM Na Phosphate, pH 6.0, 175 mM NaCl | T = 0 | 5.98 | 2.05E+13 | 91.9% |
| 3 | 10 mM Na Phosphate, pH 6.2, 175 mM NaCl | T = 0 | 6.21 | 1.77E+13 | Not Determined |
| 4 | 10 mM Na Phosphate. pH 6.5, 175 mM NaCl | T = 0 | 6.48 | 2.06E+13 | 79.5% |
| 5 | 10 mM Na Phosphate, pH 6.8, 175 mM NaCl | T = 0 | 6.82 | 1.92E+13 | 94.1% |
| 6 | 18 mM Na Phosphate, pH 7.0, 175 mM NaCl | T = 0 | 7.02 | 1.85E+13 | 97.8% |
| 1 | DPBS | 3 × FT | 7.06 | 2.04E+13 | 103.4% |
| 2 | 10 mM Na Phosphate, pH 6.0, 175 mM NaCl | 3 × FT | 6.12 | 2.08E+13 | 89.9% |
| 3 | 10 mM Na Phosphate, pH 6.2, 175 mM NaCl | 3 × FT | 6.22 | 1.72E+13 | Not Determined |
| 4 | 10 mM Na Phosphate, pH 6.5, 175 mM NaCl | 3 × FT | 6.60 | 1.89E+13 | 81.0% |
| 5 | 10 mM Na Phosphate, pH 6.2, 175 mM NaCl | 3 × FT | 6.97 | 1.90E+13 | 105.9% |
| 6 | 18 mM Na Phosphate, pH 7.0, 175 mM NaCl | 3 × FT | 7.11 | 1.29E+13 | 104.6% |
| 1 | DPBS | T = 0.5 M @ 25 C./60% RH | 7.09 | 2.02E+13 | 63.4% |
| 2 | 10 mM Na Phosphate, pH 6.0, 175 mM NaCl | T = 0.5 M @ 25 C./60% RH | 6.13 | 2.14E+13 | 35.3% |
| 3 | 10 mM Na Phosphate. pH 6.2, 175 mM NaCl | T = 0.5 M @ 25 C./60% RH | 6.39 | 1.99E+13 | Not Determined |
| 4 | 10 mM Na Phosphate, pH 6.5, 175 mM NaCl | T = 0.5 M @ 25 C./60% RH | 6.58 | 2.45E+13 | 48.5% |
| 5 | 10 mM Na Phosphate, pH 6.8, 175 mM NaCl | T = 0.5 M @ 25 C./60% RH | 6.90 | 2.48E+13 | 65.9% |
| 6 | 10 mM Na Phosphate, pH 7.0, 175 mM NaCl | T = 0.5 M @ 25 C./60% RH | 7.08 | 2.49E+13 | 85.6% |
| 1 | DPBS | T = 1 M @ 25 C./60% RH | 7.12 | 1.93E+13 | 61.5% |
| 2 | 10 mM Na Phosphate, pH 6.0, 175 mM NaCl | T = 1 M @ 25 C./60% RH | 6.19 | 1.73E+13 | 26.6% |
| 3 | 10 mM Na Phosphate, pH 6.2, 175 mM NaCl | T = 1 M @ 25 C./60% RH | 6.34 | 1.76E+13 | Not Determined |
| 4 | 10 mM Na Phosphate, pH 6.5, 175 mM NaCl | T = 1 M @ 25 C./60% RH | 6.68 | 1.94E+13 | 34.9% |
| 5 | 10 mM Na Phosphate, pH 6.8, 175 mM NaCl | T = 1 M @ 25 C./60% RH | 6.83 | 1.85E+13 | 47.3% |
| 6 | 10 mM Na Phosphate, pH 7.0, 175 mM NaCl | T = 1 M @ 25 C./60% RH | 7.04 | 2.04E+13 | 41.4% |

| | | | | Subvisible Particle Testing Results | | | | |
|---|---|---|---|---|---|---|---|---|
| | Polydispersity | % Area by | % Area by | Replicate | Particles/mL | | | |
| Form | Index² | Volume ³ | Intensity ³ | Wells | 2 μm-10 μm | ≥10 μm | ≥25 μm | ≥50 μm |
| 1 | 0.087 | 100.0 | 100.0 | 2 | 43900 | 80 | 0 | 0 |
| 2 | 0.171 | 99.9 | 97.9 | 3 | 2027 | 0 | 0 | 0 |
| 3 | 0.154 | 99.9 | 98.4 | 1 | 1720 | 40 | 0 | 0 |
| 4 | 0.100 | 100.0 | 100.0 | 2 | 27440 | 60 | 0 | 0 |
| 5 | 0.043 | 100.0 | 100.0 | 3 | 587 | 13 | 0 | 0 |
| 6 | 0.018 | 100.0 | 100.0 | 2 | 420 | 40 | 20 | 0 |
| 1 | 0.094 | 100.0 | 100.0 | 2 | 164820 | 20 | 0 | 0 |
| 2 | 0.299 | 98.9 | 83.6 | 3 | 149573 | 27 | 27 | 0 |
| 3 | 0.393 | 97.9 | 75.2 | 3 | 25052 | 27 | 0 | 0 |
| 4 | 0.169 | 99.9 | 98.0 | 2 | 26760 | 20 | 0 | 0 |
| 5 | 0.067 | 100.0 | 100.0 | 2 | 440 | 40 | 0 | 0 |
| 6 | 0.025 | 100.0 | 100.0 | 2 | 560 | 40 | 20 | 0 |
| 1 | 0.009 | 100.0 | 100.0 | 3 | 86467 | 11453 | 53 | 0 |
| 2 | 0.040 | 100.0 | 100.0 | 3 | 55427 | 453 | 0 | 0 |
| 3 | 0.061 | 100.0 | 100.0 | 3 | 114880 | 1253 | 0 | 0 |
| 4 | 0.143 | 99.9 | 99.1 | 3 | 400 | 0 | 0 | 0 |
| 5 | 0.061 | 100.0 | 100.0 | 3 | 4373 | 93 | 13 | 0 |
| 6 | 0.024 | 100.0 | 100.0 | 3 | 960 | 13 | 0 | 0 |
| 1 | 0.047 | Not Determined | 100.0 | 3 | 79987 | 7267 | 40 | 27 |
| 2 | 0.521 | Not Determined | 92.0 | 3 | 144967 | 230 | 40 | 0 |
| 3 | 0.416 | Not Determined | 90.1 | 3 | 155040 | 5907 | 40 | 13 |
| 4 | 0.261 | Not Determined | 93.5 | 3 | 480 | 27 | 0 | 0 |
| 5 | 0.047 | Not Determined | 100.0 | 3 | 5773 | 147 | 27 | 0 |
| 6 | 0.015 | Not Determined | 100.0 | 3 | 1080 | 53 | 27 | 13 |

All formulations also contain 0.005% Pluronic F68
¹T = 0.5 M @ 25 C./60% RH values for Form 4 and Form 5 represent n = 2 averages. All other niter values are n = 1
²Polydispersity index. T = 1 M @ 25 C./60% RH data collected on the Wyatt DyneProlli plate reader. All other data collected on the Malvern Zetasizer Ultra.
³ 5% Area (10-100 d · nm). T = 1 M @ 25 C./60% RH data collected on the Wyatt DyneProlli plate reader. All other data collected on the Malvern Zetasizer Ultra.

TABLE 4
Study Results

| | | | | Titer |
|---|---|---|---|---|
| Form | Description | Condition | pH | (vg/mL) |
| 1 | 10 mM Phosphate, pH 7.0, 180 mM NaCl | T = 0 | 7.11 | 7.13E+12 |
| 2 | 10 mM Phosphate, pH 7.4, 180 mM NaCl | T = 0 | 7.42 | 6.65E+12 |
| 3 | 10 mM Phosphate, pH 7.8, 120 mM NaCl | T = 0 | 7.64 | 7.18E+12 |
| 4 | 10 mM Tris, pH 7.0. 180 mM NaCl | T = 0 | 7.00 | 7.21E+17 |
| 5 | 10 mM Tris, pH 7.4, 180 mM NaCl | T = 0 | 7.36 | 6.95E+12 |
| 6 | 10 mM Tris, pH 7.8, 180 mM NaCl | T = 0 | 7.74 | 7.12E+12 |
| 1 | 10 mM Phosphate, pH 7.0, 180 mM NaCl | 3 × FT | 7.12 | 6.70E+12 |
| 2 | 10 mM Phosphate, pH 7.4, 180 mM NaCl | 3 × FT | 7.42 | 6.61E+12 |
| 3 | 10 mM Phosphate, pH 7.8, 120 mM NaCl | 3 × FT | 7.69 | 7.07E+12 |
| 4 | 10 mM Tris, pH 7.0, 180 mM NaCl | 3 × FT | 6.97 | 6.95E+13 |
| 5 | 10 mM Tris, pH 7.4, 180 mM NaCl | 3 × FT | 7.38 | 6.96E+12 |
| 6 | 10 mM Tris, pH 7.8, 180 mM NaCl | 3 × FT | 7.73 | 6.80E+12 |
| 1 | 10 mM Phosphate, pH 7.0, 180 mM NaCl | T = 2 wk @ 25 C./60% RH | 7.08 | 6.92E+12 |
| 2 | 10 mM Phosphate, pH 7.4, 180 mM NaCl | T = 2 wk @ 25 C./60% RH | 7.39 | 7.32E+12 |
| 3 | 10 mM Phosphate, pH 7.8. 180 mM NaCl | T = 2 wk @ 25 C./60% RH | 7.63 | 6.88E+12 |
| 4 | 10 mM Tris, pH 7.0, 180 mM NaCl | T = 2 wk @ 25 C./60% RH | 6.98 | 6.69E+12 |
| 5 | 10 mM Tris, pH 7.4, 180 mM NaCl | T = 2 wk @ 25 C./60% RH | 7.35 | 6.55E+12 |
| 6 | 10 mM Tris, pH 7.8. 180 mM NaCl | T = 2 wk @ 25 C./60% RH | 7.79 | 6.94E+12 |

| | | | | Subvisible Particle Testing Results | | | | |
|---|---|---|---|---|---|---|---|---|
| | Relative | | % Area by | | Particles/mL | | | |
| | Potency | Polydispersity | Intensity | Replicate | 2 μm- | ≥10 | ≥25 | ≥50 |
| Form | % | Index | (10-100 d · nm) | Wells | 10 μm | μm | μm | μm |
| 1 | 119.6% | 0.057 | 100.0 | 3 | 147 | 0 | 0 | 0 |
| 2 | 108.0% | 0.088 | 96.7 | 3 | 187 | 0 | 0 | 0 |
| 3 | 120.2% | 0.017 | 100.0 | 3 | 133 | 27 | 27 | 0 |
| 4 | 108.4% | 0.182 | 95.7 | 3 | 320 | 13 | 13 | 0 |
| 5 | 116.1% | 0.032 | 100.0 | 2 | 280 | 0 | 0 | 0 |
| 6 | 107.5% | 0.031 | 100.0 | 2 | 220 | 20 | 0 | 0 |
| 1 | 89.6% | 0.067 | 98.1 | 3 | 387 | 267 | 80 | 0 |
| 2 | 90.1% | 0.100 | 98.0 | 3 | 1133 | 53 | 13 | 13 |
| 3 | 90.1% | 0.017 | 100.0 | 2 | 140 | 0 | 0 | 0 |
| 4 | 81.8% | 0.178 | 95.8 | 3 | 133 | 0 | 0 | 0 |
| 5 | 96.5% | 0.055 | 100.0 | 2 | 100 | 0 | 0 | 0 |
| 6 | 97.0% | 0.033 | 100.0 | 2 | 380 | 0 | 0 | 0 |
| 1 | 86.1% | 0.052 | 100.0 | 3 | 3640 | 1000 | 240 | 67 |
| 2 | 80.5% | 0.072 | 100.0 | 3 | 493 | 40 | 13 | 0 |
| 3 | 86.4% | 0.018 | 100.0 | 3 | 440 | 27 | 0 | 0 |
| 4 | 66.7% | 0.322 | 92.8 | 3 | 827 | 27 | 13 | 0 |
| 5 | 90.8% | 0.120 | 97.2 | 3 | 467 | 27 | 0 | 0 |
| 6 | 98.2% | 0.082 | 98.8 | 3 | 387 | 13 | 0 | 0 |

All formulations also contain 0.005% Pluronic F68

The results of the preceding study suggested that formulating an rAAV (e.g., comprising a capsid protein of SEQ ID NO:3) in 10 mM Tris buffer, pH 7.8, 180 mM NaCl, 0.005% F68 would not only meet the primary objective (i.e., reduced subvisible particles) but might also result in improved liquid storage stability. Therefore, pH 7.8 was revisited (in Phosphate, Tris, and Hepes) and compared against Form 1 (DPBS, 0.005% F68) during storage at 25° C./60% RH. Relative potency results can be found in Table 5. All four formulations behaved very similarly, possibly with subtle improvements associated with pH 7.8 formulations (Form 1-3) after 2-week storage @25° C./60% RH. Additionally, mass spectrometry results (Table 6) revealed no new degradants for the pH 7.8 formulations at T=0. However, Phosphate and Hepes pH 7.8 formulations did appear to show an increased rate of deamidation relative to Form 1 during 2-week storage @25 C/60% RH. Conversely, the Tris, pH 7.8 formulation showed a deamidation profile that was very similar to Form 1 while also exhibiting a reduced risk of oxidation.

TABLE 5
Relative Potency (% RP) Summary

| | | | | Titer | Relative | Relative |
|---|---|---|---|---|---|---|
| Form | Description | Condition | pH | (vg/mL) | Potency %¹ | Potency %² |
| 1 | 10 mM Phosphate, pH 7.8, 180 mM NaCl | T = 0 | 7.65 | 1.78E+12 | 120.0% | 98.4% |
| 2 | 10 mM Tris, pH 7.8, 180 mM NaCl | T = 0 | 7.84 | 1.79E+12 | 121.4% | 99.5% |
| 3 | Hepes, pH 7.8, 180 mM NaCl | T = 0 | 7.87 | 1.69E+12 | 122.0% | 105.6% |
| 4 | DPBS | T = 0 | 6.97 | 1.75E+12 | 135.0% | 100.0% |
| 1 | 10 mM Phosphate, pH 7.8, 180 mM NaCl | T = 2 wk @ 25 C./60% RH | 7.63 | 1.57E+12 | 89.1% | 80.8% |
| 2 | 10 mM Tris, pH 7.8, 180 mM NaCl | T = 2 wk @ 25 C./60% RH | 7.81 | 1.44E+12 | 90.5% | 77.6% |
| 3 | Hepes, pH 7.8, 180 mM NaCl | T = 2 wk @ 25 C./60% RH | 7.78 | 1.59E+12 | 93.5% | 76.6% |
| 4 | DPBS | T = 2 wk @ 25 C./60% RH | 7.01 | 1.56E+12 | 85.8% | 72.6% |
| 1 | 10 mM Phosphate, pH 7.8, 180 mM NaCl | T = 1 M @ 25 C./60% RH | 7.67 | 1.82E+12 | 83.4% | 59.4% |
| 2 | 10 mM Tris, pH 7.8, 180 mM NaCl | T = 1 M @ 25 C./60% RH | 7.86 | 1.70E+12 | 79.3% | 65.5% |
| 3 | Hepes, pH 7.8, 180 mM NaCl | T = 1 M @ 25 C./60% RH | 7.83 | 1.75E+12 | 78.6% | 66.8% |
| 4 | DPBS | T = 1 M @ 25 C./60% RH | 7.06 | 1.81E+12 | 76.1% | 65.4% |

All formulations also contain 0.005% Pluronic F68
¹N = 3 (From Plates 1-3), calculated relative to 40-150 reference standard
²N = 1 (From Plate 4), calculated relative to Form 4, T = 0

TABLE 6
Mass Spectrometry Results

| | | | | Form 1 | | Form 2 | |
|---|---|---|---|---|---|---|---|
| | | | | 10 mM Phosphate, pH 7.8, 180 | | 10 mM Tris, pH 7.8, 180 mM | |
| | | | | mM NaCl, 0.005% F68 | | NaCl, 0.005% F68 | |
| | Start | | | | T = 2 wk | | T = 2 wk |
| Sites | AA | Sequence | Modifications | T = 0 | @ 25 C./60% RH | T = 0 | @ 25 C./60% RH |
| Ri57n | 52 | [symbol] | n: De [symbol] | 4.10% | 9.50% | 2.70% | 5.80% |
| N223n | 219 | [symbol] | n: De [symbol] | 2.80% | 2.60% | 0.70% | 0.70% |
| N253 | 246 | [symbol] | n: De [symbol] | 1.40% | 1.50% | 1.80% | 1.80% |
| or 254n | | | | | | | |
| N476n | 476 | [symbol] | n: De [symbol] | 0.60% | 1.10% | 0.30% | 0.20% |
| N495 or 496 or | 492 | [symbol] | n: De [symbol] | 4.20% | 7.20% | 2.00% | 3.50% |
| 497n | | | | | | | |
| M434m | 433 | [symbol] | m: Oxidized | 4.20% | 4.40% | 1.30% | 1.20% |
| | | | methionine | | | | |
| M558m | 557 | [symbol] | m: Oxidized | 0.90% | 1.10% | 0.40% | 0.40% |
| | | | methionine | | | | |
| M614m | 599 | [symbol] | m: Oxidized | 1.40% | 3.10% | 0.70% | 0.70% |
| | | | methionine | | | | |
| S149s | 143 | [symbol] | [symbol] phosphorylated | 2.70% | 2.70% | 2.40% | 9.10% |
| | | Total | De [symbol] | 18.10% | 22.00% | 7.50% | 12.00% |
| | | | Oxidation | 6.50% | 7.60% | 2.40% | 2.30% |
| | | | Phosphorylation | 2.70% | 2.70% | 2.40% | [symbol] .10% |

| | Form 3 | | | |
|---|---|---|---|---|
| | 10 mM Hepes, pH 7.8, 180 mM | | Form 4 | |
| | NaCl, 0.005% F68 | | DPBS, 0.005% F68 | |
| | | T = 2 wk | | T = 2 wk |
| Sites | T = 0 | @ 25 C./60% RH | T = 0 | @ 25 C./60% RH |
| Ri57n | 3.60% | 8.40% | 3.00% | 4.10% |
| N223n | 1.30% | 3.00% | 1.10% | 3.90% |
| N253 | 2.30% | 3.00% | 1.60% | 1.70% |
| or 254n | | | | |
| N476n | 0.50% | 1.10% | 0.60% | 0.60% |
| N495 or 496 or | 2.90% | 4.60% | 2.50% | 4.20% |
| 497n | | | | |
| M434m | 3.00% | 5.60% | 2. [symbol] | 4.50% |
| M558m | 0.60% | 1.30% | 0. [symbol] | 0.90% |
| M614m | 1.50% | 2.50% | 1.10% | 1.70% |
| S149s | 2.50% | 2.60% | 2.80% | 2.70% |
| | [symbol] | [symbol] | 8.80% | 12.50% |
| | 5.20% | 9.40% | 4.10% | 7. [symbol] 0% |
| | 3.50% | 2.50% | [symbol] | 2.70% |

[symbol]

indicates data missing or illegible when filed

### Confirmatory Forced Degradation Studies

Collectively, subvisible particle data, relative potency and chemical stability all suggested that a Tris based buffer would make a suitable choice for an improved rAAV formulation. Therefore, the intent of the next formulation development study was to perform a bracketed (Tris, pH 7.8±0.3) forced degradation experiment in a representative container closure system (i.e., 0.3 mL fill in a 2 mL CZ vial). Samples were tested at T=0, after 3 freeze-thaw cycles (3×FT), and after 2-week storage at 2-8° C. and 25° C./60% RH. Results can be found in Table 8. The FDA requires “GT vector-based final products should be tested for particulate matter, and the test method and release criteria should follow USP <789>.” See https://fda.gov/media/124641/download Subvisible particle testing yielded results that met USP <789> limits for 11 of 12 tested samples. Additionally, 100% area (10-100 d·nm. by Volume) was observed for 11 of 12 tested samples. These two results appeared to confirm increased physical stability within the range tested. Relative potency also proved to be very similar for all three formulations. Each showed a reduction in aflibercept expression after 2 week storage at 25° C./60% RH and >100% relative potency after 2 week storage at 2-8° C.

TABLE 7
USP <789> Subvisible Particle Test Limits

| | Diameter | | |
|---|---|---|---|
| | ≥10 μm | ≥25 μm | ≥50 μm |
| Number of Particles | 50/mL | 5/mL | 2/mL |

TABLE 8
Results Summary

| | | | | | | Poly- | % Area by | Subvisible Particle Testing Results | | | | |
|---|---|---|---|---|---|---|---|---|---|---|---|---|
| | | | | | Relative | dis- | Volume | Re- | | | | |
| | | | | Titer | Potency | persity | (10-100 | plicate | Particles/mL | | | |
| Form | Description | Condition | pH² | (ug/mL) | % | Index | d · nm) | [symbol] | ≥2 μm | ≥10 μm | ≥25 μm | ≥50 μm |
| 1 | 10 mM Tris, pH 7.8, | T = 0 | 7.78 | 1.52E+12 | 115.9% | 0.128 | 100.0 | 3 | 433 | 17 | 17 | 0 |
| | 180 mM NaCl | | | | | | | | | | | |
| 2 | 10 mM Tris, pH 8.0, | T = 0 | 7.98 | 2.07E+12 | 112.3% | 0.078 | 100.0 | 0 | 333 | 0 | 0 | 0 |
| | 180 mM NaCl | | | | | | | | | | | |
| 3 | 10 mM Tris, pH 8.3, | T = 0 | 8.30 | 1.45E+12 | 105.8% | 0.162 | 99.3 | 0 | 267 | 0 | 0 | 0 |
| | 180 mM NaCl | | | | | | | | | | | |
| 1 | 10 MM Tris, pH 7.8, | 3XFT | Not | 1.49E+12 | Not | 0.018 | 100.0 | 0 | 1100 | 17 | 0 | 0 |
| | 180 mM NaCl | | Tested | | Tested | | | | | | | |
| 2 | 10 mM Tris, pH 8.0, | 3XFT | Not | 1.99E+12 | Not | 0.105 | 100.0 | 0 | [symbol] | 0 | 0 | 0 |
| | 180 mM NaCl | | Tested | | Tested | | | | | | | |
| 1 | 10 mM Tris, pH 8.3, | 3XFT | Not | 1.52E+12 | Not | 0.096 | 106.0 | 0 | 467 | 0 | 0 | 0 |
| | 186 mM NaCl | | Tested | | Tested | | | | | | | |
| 1 | 10 mM Tris, pH 7.8, | T = 2 wk | Not | 1.52E+12 | 101.4% | 0.018 | 106.0 | 0 | 423 | 0 | 0 | 0 |
| | 128 mM NaCl | @ 2-8° C. | Tested | | | | | | | | | |
| 2 | 10 mM Tris, pH 8.0, | T = 2 wk | Not | 2.07E+12 | 102.6% | 0.031 | 100.0 | 3 | 317 | 0 | 0 | 0 |
| | 180 mM NaCl | @ 2-8° C. | Tested | | | | | | | | | |
| 3 | 10 mM Tris, pH 8.3, | T = 2 wk | Not | 1.47E+12 | 100.8% | 0.012 | 100.0 | 3 | 250 | 0 | 0 | 0 |
| | 120 mM NaCl | @ 2-8° C. | Tested | | | | | | | | | |
| 1 | 10 mM Tris, pH 7.8, | T = 2 wk @ | Not | 1.47E+12 | 86.6% | 0.042 | 100.0 | 3 | 400 | 0 | 0 | 0 |
| | 180 mM NaCl | 25° C./60% RH | Tested | | | | | | | | | |
| 2 | 10 mM Tris, pH 8.0, | T = 2 wk @ | Not | 1.54E+12 | 79.3% | 0.020 | 100.0 | 3 | 867 | 33 | 0 | 0 |
| | 180 mM NaCl | 25° C./60% RH | Tested | | | | | | | | | |
| 3 | 10 mM Tris, pH 8.3, | T = 2 wk @ | Not | 1.58E+12 | 81.7% | 0.012 | 100.0 | 3 | 467 | 0 | 0 | 0 |
| | 180 mM NaCl | 25° C./60% RH | Tested | | | | | | | | | |

All formulations also contain 0.005% Pluronic F68
*pH testing was performed on a separate vial that was treated similar to T = 0.
Also noting that sample pH was measured after room temperature equilibration (−20.4° C.) [symbol]
indicates data missing or illegible when filed

This confirmatory development study results ultimately led to the nomination of the formulation which was initially described as 10 mM Tri s buffer, pH 8.0, 180 mM NaCl, 0.0050% F68. However, it was noted that most pH testing conducted during formulation development was measured at room temperature (typically ranging from −19-21° C.). Therefore, in an effort to improve the robustness of the diluent manufacture (specifically the known temperature dependence exhibited by Tris buffer) it was decided that all future pH testing (in-process and at release) should be conducted at 25° C. in accordance with USP <791>. Therefore, three preparations of the lead formulation buffer were prepared, and pH tested at 20° C. and 25° C. (Table 8). As expected, pH measurement at 25° C. yielded a drop in pH of approximately 0.1 pH unit. This prompted a corresponding change in target pH (7.9±0.3) as well as buffer description (10 mM Tris buffer, pH 7.9, 180 mM NaCl, 0.005% F68).

TABLE 8
Lead Formulation Buffer pH Measured at 20° C. and 25° C.

| | pH | Temperature (C°) | pH | Temperature (C°) |
|---|---|---|---|---|
| Prep 1 | 8.01 | 20.0 | 7.87 | 24.9 |
| Prep 2 | 8.01 | 20.0 | 7.87 | 25.1 |
| Prep 3 | 8.01 | 19.9 | 7.86 | 25.1 |
| Average | 8.01 | 20.0 | 7.87 | 25.0 |
| Std DEV | 0.000 | 0.06 | 0.006 | 0.113 |
| % RSD | 0.0% | 0.3% | 0.1% | 0.5% |

### Example 2

The impact of sodium chloride concentration (140-200 mM) was evaluated in the below formulations at T=0 and after 3 freeze-thaw cycles (3×FT). All three formulations contained rAAV, comprising a capsid protein of SEQ ID NO:3 and a nucleic acid comprising the nucleotide sequence of SEQ ID NO:6, at a target concentration of ~1.6E12 vg/mL
20 mM Tris, pH 8¹, 140 mM NaCl, 0.005% F68
20 mM Tris, pH 8¹, 180 mM NaCl, 0.005% F68
20 mM Tris, pH 8¹, 200 mM NaCl, 0.005% F68 ¹ pH 8 is the target pH at room temperature. This is expected to be approximately 0.1 pH unit lower (i.e., 7.9) when measured at 25 C

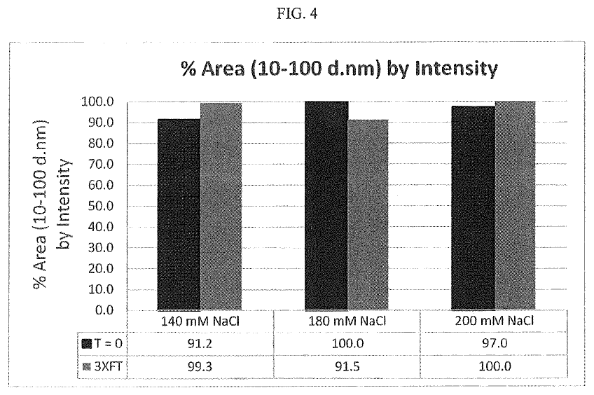

Sample testing on a Wyatt DynaPro III DLS Plate Reader (FIG. 4) demonstrated similar colloidal stability, i.e., >90% Area (10-100 d·nm) by Intensity, across the range of sodium chloride evaluated.

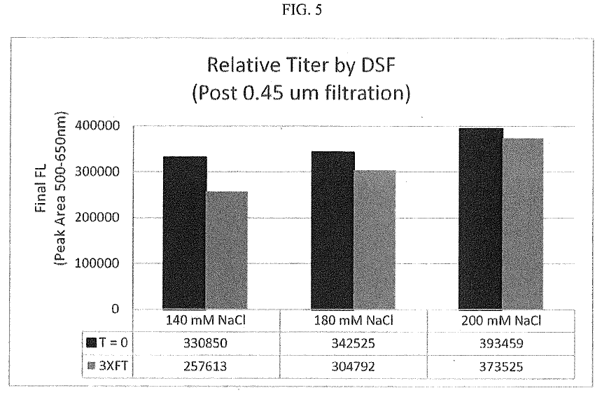

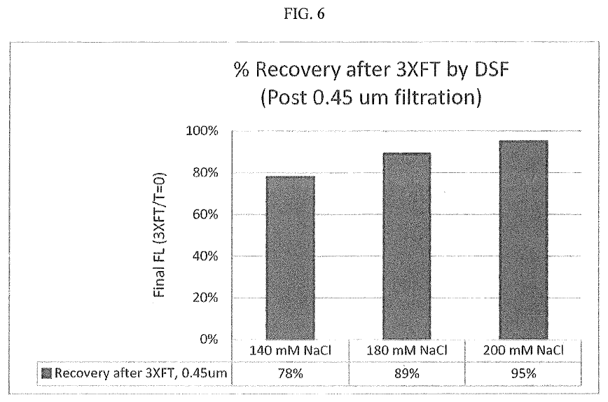

Samples were, subsequently, 0.45 um filtered, spiked with 20× Sybr Gold and tested by Differential Scanning Fluorimetry (Uncle Instrument by Unchained Labs). Final Fluorescence (Final FL) values reported in FIG. 5 represent the fluorescent emission signal ranging from 500-650 nm collected at 15° C., after samples were subjected to a thermal melt (15-95° C.). Results suggest similar amounts of accessible ssDNA (i.e., relative titer) present in all three formulations at T=0.3×FT samples showed slightly higher signal recoveries for sodium chloride concentrations ≥180 mM NaCl. However, none of the filtered, 3×FT stressed samples showed a recovery of less than 78% (FIG. 6).

Many of the study results above contain pH values which were measured at room temperature. However, as described in Table 8, Tris buffer exhibits temperature dependence. This temperature dependence has also been captured in a separate study in which a Tris, pH 7.8 buffer was measured at room temperature (the same temperature it was initially made and tested at) and after equilibration in either a 2-8° C. or a 25° C./60% RH stability chamber. The results indicate that a pH shift of approximately −0.1 pH unit should be expected for a 10 mM Tris buffer prepared at room temperature and then stored at 25° C. (consistent with Table 8). Additionally, a 0.3 pH unit shift is expected for 10 mM Tris buffer prepared at room temperature and then stored at 2-8° C. This, in turn, implies that rAAV comprising a capsid protein of SEQ ID NO:3 has shown stability in Tris buffered saline solutions ranging from approximately pH 7.3 (pH 7.4 formulation stored at 25 C/60% RH) to 8.6 (pH 8.3 formulation stored at 2-8 C) as shown in Table 9 below.

TABLE 9
Tris Buffer Temperature Dependence

| | 2-8° C. | | Room Temp. | 25° C./60% RH | |
|---|---|---|---|---|---|
| Buffer | pH | ΔpH¹ | pH | pH | ΔpH¹ |
| 10 mM Tris, | 8.179 | 0.323 | 7.856 | 7.751 | −0.105 |
| pH 7.8, 180 mM | | | | | |
| NaCl, 0.0056 F68 | | | | | |

¹change in pH relative to pH at room temperature

### Example 3

The long-term stability of rAAV, comprising a capsid protein of SEQ ID NO:3 and a nucleic acid comprising the nucleotide sequence of SEQ ID NO:6, formulated in 10 mM Tris, 180 mM NaCl, 0.00500 Pluronic F68 has, subsequently, been evaluated at both s −60° C. and 2-8° C.

In short, product development lots 4DER000096 and 4DER000097 (2-mL CZ vial filled with rAAV, comprising a capsid protein of SEQ ID NO:3 and a nucleic acid comprising the nucleotide sequence of SEQ ID NO:6, formulated in 10 mM Tris, 180 mM NaCl, 0.0050 Pluronic F68), were evaluated in an abbreviated long-term stability study conducted at <−60° C. and 2-8° C. pH, subvisible particles, genomic titer, transgene expression (protein-based) and monodispersion results can be found in Table 10 and Table 11 below. Results for clinical lot 4D32210031 can be found in Table 12.

TABLE 10
Stability at ≤−60° C. and 2-8° C.

| Storage at ≤−60° C. | | Test Interval (Months) | | |
|---|---|---|---|---|
| Attribute | Test Method | 0 | 3 | 12 |
| pH | Potentiometry | 7.6 | 7.5 | 7.5 |
| Subvisible Particles | Background Membrane | ≥10 μm: 0 | ≥10 μm: 0 | ≥10 μm: 0 |
| | Imaging ( Particles/mL) | ≥25 μm: 0 | ≥25 μm: 0 | ≥25 μm: 0 |
| | | ≥50 μm: 0 | ≥50 μm: 0 | ≥50 μm: 0 |
| Genomic Titer | ddPCR (vg/mL) | 1.99E+12 | 2.12E+12 | 1.98E+12 |
| Transgene Expression | Cell Transduction Assay | 121.8 | 142.6 | 125.9 |
| (Protein-based) | with Expression by ELISA | | | |
| | (% Relative Potency) | | | |
| Monodispersion | DLS (% 10-100 d · nm) | 100 | 100 | 100 |
| Storage at 2-8° C. | | Test Interval (Months) | | |
| Attribute | Test Method | 0 | 3 | 6 |
| pH | Potentiometry | 7.6 | 7.5 | 7.5 |
| Subvisible Particles | Background Membrane | ≥10 μm: 0 | ≥10 μm: 10 | ≥10 μm: 0 |
| | Imaging (Particles/mL) | ≥25 μm: 0 | ≥25 μm: 0 | ≥25 μm: 0 |
| | | ≥50 μm: 0 | ≥50 μm: 0 | ≥50 μm: 0 |
| Genomic Titer | ddPck (vg/mL) | 1.99E+12 | 2.15E+12 | 1.95E+12 |
| Transgene Expression | Cell Transduction Assay | 114.6 | 113.7 | 100 |
| (Protein-based) | with Expression by ELISA | | | |
| | (% Relative Potency) | | | |
| Monodispersion | DLS (% 10-100 d · nm) | 100 | 100 | 100 |

TABLE 11
Stability at ≤−60° C. and 2-8° C.

| Storage at ≤−60° C. | | Test Interval (Months) | | |
|---|---|---|---|---|
| Attribute | Test Method | 0 | 3 | 12 |
| pH | Potentiometry | 7.9 | 7.8 | 7.8 |
| Subvisible Particles | Background Membrane | ≥10 μm: 0 | ≥10 μm: 0 | ≥10 μm: 0 |
| | Imaging (Particles/mL) | ≥25 μm: 0 | ≥25 μm: 0 | ≥25 μm: 0 |
| | | ≥50 μm: 0 | ≥50 μm: 0 | ≥50 μm: 0 |
| Genomic Titer | ddPCR (vg/ml) | 1.73E+12 | 2.008+12 | 1.778+12 |
| Transgene Expression | Cell Transduction Assay | 122.1 | 141.9 | 124.3 |
| (Protein-based) | with Expression by ELISA | | | |
| | (% Relative Potency) | | | |
| Monodispersion | DLS (% 10-100 d · nm) | 100 | 100 | 100 |
| Storage at 2-8° C. | | Test Interval (Months) | | |
| Attribute | Test Method | 0 | 3 | 6 |
| pH | Potentiometry | 7.9 | 7.8 | 7.8 |
| Subvisible Particles | Background Membrane | ≥10 μm: 0 | ≥10 μm: 0 | ≥10 μm: 10 |
| | Imaging (Particles/mL) | ≥25 μm: 0 | ≥25 μm: 0 | ≥25 μm: 0 |
| | | ≥50 μm: 0 | ≥50 μm: 0 | ≥50 μm: 0 |
| Genomic Titer | ddPCR (vg/mL) | 1.73E+12 | 1.98E+12 | 1.718+12 |
| Transgene Expression | Cell Transduction Assay | 122.1 | 129 | 89.6 |
| (Protein-based) | with Expression by ELISA | | | |
| | (% Relative Potency) | | | |
| Monodispersion | DLS (% 10-100 d · nm) | 100 | 100 | 100 |

TABLE 12
Clinical Lot Stability at ≤−60° C. and 2-8° C.

| Storage at ≤−60° C. | | Test Interval (Months) | | | |
|---|---|---|---|---|---|
| Attribute | Test Method | 0 | 3 | 6 | 12 |
| pH | Potentiometry | 7.9 | 7.8 | 7.8 | 7.8 |
| Subvisible Particles | Background Membrane | ≥10 μm: 2 | ≥10 μm: 0 | Not Tested | ≥10 μm: 0 |
| | imaging (Particles/mL) | ≥25 μm: 0 | ≥25 μm: 0 | | ≥25 μm: 0 |
| | | ≥50 μm: 0 | ≥50 μm: 0 | | ≥50 μm: 0 |
| Genomic Titer | ddPCR (vg/ml) | 1.90E+12 | 2.20E+12 | 1.90E+12 | 1.90E+12 |
| Transgene Expression | Cell Transduction Assay | 123.7 | 110.1 | 88.8 | 107.7 |
| (Protein-based) | with Expression by ELISA | | | | |
| | (% Relative Potency) | | | | |
| Monodispersion | DLS (% 10-100 d · nm) | 100 | 100 | 100 | 100 |
| Storage at 2-8° C. | | Test Interval (Months) | | | |
| Attribute | Test Method | 0 | 3 | 6 | 12 |
| pH | Potentiometry | 7.7 | Not Tested | 7.6 | Not Tested |
| Subvisible Particles | Background Membrane | ≥10 μm: 2 | ≥10 μm: 0 | Not Tested | ≥10 μm: 30 |
| | Imaging (Particles/mL) | ≥25 μm: 0 | ≥25 μm: 0 | | ≥25 μm: 0 |
| | | ≥50 μm: 0 | ≥50 μm: 0 | | ≥50 μm: 0 |
| Genomic Titer | ddPCR (vg/mL) | 1.90E+12 | 2.20E+12 | 2.00E+12 | 2.00E+12 |
| Transgene Expression | Cell Transduction Assay | 114.9 | 107.9 | 84.6 | 82.3 |
| (Protein-based) | with Expression by ELISA | | | | |
| | (% Relative Potency) | | | | |
| Monodispersion | DLS (% 10-100 d · nm) | 100 | 100 | 100 | 100 |

Results indicate rAAV, comprising a capsid protein of SEQ ID NO:3 and a nucleic acid comprising the nucleotide sequence of SEQ ID NO:6, formulated in 10 mM Tris, 180 mM NaCL, 0.005% Pluronic F68 is stable for more than 12-months at ≤−60° C. and equal or greater than 3 months at 2-8° C.

| Attribute | Value | Reference |
|---|---|---|
| Vitreous Chamber | 4.649.99 ± 0.426.54 | Azhdam, Ariel M., et |
| Volume | mm³ (women) | al. Vis. Sci. Tech. |
| (In-Vivo) | 4.969.0 ± 0.465.20 | 2020; 9(1):2. |
| | mm³ (men) | |
| Osmolality (living | 289.5 ± 6.9 mOsm/kg | Kokavec J., et al. Clin |
| human vitreous) | | Exp Ophthalmol. 2016 |
| | | September; 44(7):597-609 |
| pH (fresh vitreous | 7.29 with a 95% CI | Sobolewska et al. Int J |
| samples) | of 7.20-7.39 | Retin Vitr (2017) 3:22 |

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention.

1. (canceled)

2. (canceled)

3. A pharmaceutical composition comprising adeno-associated virus (AAV) comprising a capsid of serotype 2 (AAV2) or a variant thereof and a buffering composition, said buffering composition comprising:
(i) about 7 mM to about 12 mM of a Tris buffer;
(ii) about 150 to about 200 mM of a pharmaceutically acceptable salt; and
(iii) about 0.0005% (w/v) to about 0.01% (w/v) of a non-ionic surfactant;
wherein the buffering composition has a pH above 7.0 and below about 8.5 and wherein the pharmaceutical composition does not comprise a divalent cation and does not comprise a sugar or sugar alcohol.

4. The pharmaceutical composition according to claim 3, wherein the buffering composition
has a pH of about 7.5 to about 8.3.

5. (canceled)

6. The pharmaceutical composition to claim 3, wherein the buffering composition comprises:
(i) about 10 mM of a Tris buffer;
(ii) about 180 mM of the pharmaceutically acceptable salt;
(iii) about 0.005% of the nonionic surfactant; and
wherein the buffering composition has a pH of about 7.9.

7. (canceled)

8. The pharmaceutical composition according to claim 3, wherein the pharmaceutical composition comprises about 7 mM, about 8 mM, about 9 mM, about 10 mM, about 11 mM, or about 12 mM Tris buffer.

9. The pharmaceutical composition according to claim 3 wherein the pharmaceutically acceptable salt is selected from the group consisting of a sodium salt, an ammonium salt, a potassium salt and a combination thereof.

10. The pharmaceutical composition according to claim 9, wherein the pharmaceutically acceptable salt is NaCl.

11. The pharmaceutical composition according to claim 10, comprising about about 160-200 mM, about 170-190 mM, about 175-190 mM, about 175-185 mM or about 180 mM NaCl.

12. The pharmaceutical composition according to claim 3, wherein the buffering composition has a pH of about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.

13. The pharmaceutical composition according claim 3, wherein the nonionic surfactant is Pluronic F68, wherein the pharmaceutical composition comprises about 0.001%, about 0.002%, about 0.003%, about 0.004%, about 0.005%, about 0.006%, about 0.007%, about 0.008%, about 0.009% or about 0.01% Pluronic F68.

14. The pharmaceutical composition according to claim 3, wherein the pharmaceutical composition exhibits reduced subvisible particle formation and/or improved liquid storage stability compared to a pharmaceutical composition comprising phosphate-buffered saline (PBS) buffer (8 mM Na2HPO4, 1.5 mM KH2PO4, 2.7 mM KCl, 138 mM NaCl, 0.9 mM CaCl2 and 0.5 mM MgCl2, pH 7.0; DPBS) and Pluronic F68.

15. (canceled)

16. (canceled)

17. (canceled)

18. (canceled)

19. (canceled)

20. The pharmaceutical composition according to claim 3, wherein the AAV comprises a variant capsid protein having an amino acid sequence that is at least 90% identical, at least 95% identical, at least 98% identical or 100% identical to the entire length of the amino acid sequence set forth as SEQ ID NO:3.

21. The pharmaceutical composition according to claim 20, wherein the AAV is a recombinant AAV (rAAV) comprising a heterologous nucleic acid comprising a nucleotide sequence encoding a gene product, wherein the gene product is an interfering RNA and/or a polypeptide and wherein the nucleotide sequence encoding the gene product is operably linked to a promoter.

22. (canceled)

23. The pharmaceutical composition according to claim 21, wherein the heterologous nucleic acid comprises a nucleotide sequence encoding a polypeptide of SEQ ID NO:5.

24. The pharmaceutical composition according to claim 23, wherein the nucleotide sequence encoding a polypeptide of SEQ ID NO:5 is at least 80%, at least 85%, at last 90%, at least 95%, or is 100% identical to SEQ ID NO:4.

25. (canceled)

26. The pharmaceutical composition according to claim 24, wherein the heterologous nucleic acid further comprises a nucleotide sequence at least 70% identical to the nucleotide sequence set forth as SEQ ID NO:6.

27. (canceled)

28. (canceled)

29. (canceled)

30. (canceled)

31. (canceled)

32. (canceled)

33. (canceled)

34. The pharmaceutical composition according to claim 21, wherein the heterologous nucleic acid comprises a nucleotide sequence encoding a complement regulator factor H (CFH) protein or a fragment thereof.

35. The pharmaceutical composition according to claim 34, wherein the nucleotide sequence encoding the CFH protein or fragment thereof has at least 80%, at least 85%, at least 90%, at least 95%, or at least 98% sequence identity to the amino acid sequence set forth in SEQ ID NO:9.

36. The pharmaceutical composition according to claim 35, wherein the nucleotide sequence encoding the CFH protein or fragment thereof comprises the amino acid sequence set forth in SEQ ID NO:9.

37. The pharmaceutical composition according to claim 36, wherein the rAAV comprises a heterologous nucleic acid comprising a nucleotide sequence with at least 80%, at least 85%, at least 90%, at least 95%, at least 98% sequence identity to the nucleotide sequence set forth in any one of SEQ ID NOs:10-13 or is identical to the nucleotide sequence set forth in any one of SEQ ID Nos:10-13.

38. (canceled)

39. The pharmaceutical composition according to claim 21 wherein the nucleic acid further comprises a 5′ ITR, a promoter operably linked to said nucleotide sequence, a polyadenylation sequence and a 3′ ITR.

40. The pharmaceutical composition according to claim 39, wherein the promoter is a ubiquitous promoter, preferably wherein the promoter is a CAG or CBA promoter.

41. (canceled)

42. The pharmaceutical composition according to claim 3, wherein the pharmaceutical composition comprises a unit dose of 1×10¹⁰ vector particles (vp) to 1×10¹³ vp or between 1×10¹¹ to 1×10¹² vp.

43. The pharmaceutical composition according to claim 3, wherein the pharmaceutical composition is a storage stable composition.

44. The pharmaceutical composition according to claim 43, wherein the pharmaceutical composition is stable as a liquid for at least 1, 2, 3, 4, 5, 6, 7, 8, 9, 10, 11, 12, 13, 14, 15, 16, 17, 18, 19, 20, 21, 22, 23, or at least 24 months at a temperature of less than or equal to about −60° C.

45. The pharmaceutical composition according to claim 44, wherein the pharmaceutical composition is stable as a liquid for at least two weeks, at least 3 weeks, at least one month, at least 5 weeks, at least 6 weeks, at least 7 weeks, at least 2 months, at least 9 weeks, at least 10 weeks, at least 11 weeks, at least 3 months, at least 13-weeks, at least 14 weeks, at least 15 weeks, at least 4 months, at least 17 weeks, at least 18 weeks, at least 19 weeks, at least 5 months or at least 6 months at a temperature of about 2-8° C.

46. The pharmaceutical composition according to claim 45, wherein the pharmaceutical composition maintains a relative potency in liquid form of at least 90%, at least 95%, at least 96%, at least 97%, at least 98% or at least 99% for at least 1, 2, 3, 4, 5, 6, 7, 8, 9, 10, 11, 12, 13, 14, 15, 16, 17, 18, 19, 20, 21, 22, 23 or at least 24 months.

47. The pharmaceutical composition according to claim 26, wherein the buffering composition comprises about 10 mM Tris, about 180 mM NaCL, about 0.005% Pluronic F68 and wherein the pH of the buffering composition is about 7.4° C. to about 8.4 at 25° C.

48. The pharmaceutical composition according to claim 47, wherein the pH of the buffering composition is about 7.6° C. to about 8.2 at 25° C.

49. (canceled)
